(19) United States
(12) Patent Application Publication
(10) Pub. No.: US 2022/0268762 A1
(43) Pub. Date: Aug. 25, 2022

(54) METHODS, SYSTEMS, AND COMPUTER-READABLE MEDIA FOR PREDICTING A CANCER PATIENT'S RESPONSE TO IMMUNE-BASED OR TARGETED THERAPY

(71) Applicant: H. LEE MOFFITT CANCER CENTER AND RESEARCH INSTITUTE, INC., Tampa, FL (US)

(72) Inventors: Frederick L. LOCKE, Lutz, FL (US); Philipp Martin Friedhelm ALTROCK, Tampa, FL (US); Gregory J. KIMMEL, Ocoee, FL (US)

(21) Appl. No.: 17/629,827

(22) PCT Filed: Jul. 28, 2020
(86) PCT No.: PCT/US2020/043870

§ 371 Date: Jan. 25, 2022

Related U.S. Application Data
(60) Provisional application No. 62/879,534, filed on Jul. 28, 2019.

(51) Int. Cl. G01N 33/50; G01N 33/574; G06N 20/00; G16H 50/20

(52) U.S. Cl. CPC G01N 33/5091; G16H 50/20; G06N 20/00; G01N 33/574

(57) ABSTRACT

Methods, systems, and computer-readable media for predicting a patient's response to immune based or target therapy are described herein. An example computer-implemented method includes generating a model configured to represent dynamics and interactions among normal T cells, engineered cells, and tumor cells, where the model includes a plurality of cell population compartments. The computer-implemented method also includes receiving pre-treatment patient data for a cancer patient, and receiving post-treatment patient data for the cancer patient. Each of the pre-treatment patient data and the post-treatment patient data includes a measure of at least one of tumor volume, total lymphocytes, memory T cells, memory engineered cells, tumor killing cells, or antigen-presenting tumor cells. The computer-implemented method further includes quantitatively predicting the cancer patients response to the immune-based or targeted therapy using the model, the pre-treatment patient data, and the post-treatment patient data.

### CROSS-REFERENCE TO RELATED APPLICATIONS

This application claims the benefit of U.S. provisional patent application No. 62/879,534, filed on Jul. 28, 2019, and entitled “METHODS OF ENHANCING CAR T CELL THERAPY,” the disclosure of which is expressly incorporated herein by reference in its entirety.

### BACKGROUND

Large B cell Lymphoma (LBCL) is curable in a majority of patients treated with upfront chemoimmunotherapy. Historically, the prognosis for patients refractory to first or second line therapy is dismal, with only 7% able to achieve a complete response to the next line of chemotherapy and a median overall survival of less than 7 months. These patients can now benefit from Chimeric Antigen Receptor (CAR) T cell therapies, such as axicabtagene ciloleucel (axi-cel) which consists of autologous engineered T cells re-targeted against the CD19+ lymphoma. Several factors are known to be associated with efficacy of CAR T cell therapy, including the size of lymphoma tumor mass, the degree of T cell expansion, the number CAR T cells with a less differentiated phenotype within the infusion product, and the degree of lymphodepletion provided by conditioning chemotherapy prior to CAR T infusion. What is needed are mechanistic understandings of how these individual processes relate to each other and to the desired outcome of long-term durable remission following CAR T therapy. Specifically, what is needed are new methods of treating cancer that takes into account these factors to establish long-term remission.

### SUMMARY

Disclosed are methods, systems, and computer-readable media related to immune-based or targeted therapy.

In one aspect, an example computer-implemented method is described. The computer-implemented method includes generating a model configured to represent dynamics and interactions among normal T cells, engineered cells, and tumor cells, where the model includes a plurality of cell population compartments. The computer-implemented method also includes receiving pre-treatment patient data for a cancer patient, and receiving post-treatment patient data for the cancer patient. Each of the pre-treatment patient data and the post-treatment patient data includes a measure of at least one of tumor volume, total lymphocytes, memory T cells, memory engineered cells, tumor killing cells, or antigen-presenting tumor cells. The computer-implemented method further includes quantitatively predicting the cancer patient's response to an immune-based or targeted therapy using the model, the pre-treatment patient data, and the post-treatment patient data.

Additionally, the model is configured to simulate interactions between normal T cells and engineered cells.

Alternatively or additionally, the model is configured to simulate a differentiation rate of memory engineered cells to tumor killing cells.

Alternatively or additionally, the plurality of cell population compartments include normal naïve/memory T cells, naïve/memory engineered cells, tumor killing cells, and antigen-presenting tumor cells. In these implementations, the plurality of cell population compartments can be modelled based on continuous-time birth and death stochastic processes and deterministic mean-field equations.

Optionally, in some implementations, the post-treatment patient data further includes a measure of at least one tumor growth rate, tumor cell extinction rate, memory T cell recovery rate, naïve/memory engineered cell expansion rate, naïve/memory engineered cell differentiation rate, tumor killing cell death rate, or tumor killing cell exhaustion rate.

In some implementations, the quantitative prediction of the cancer patient's response to the immune-based or targeted therapy is a probability of tumor extinction. Optionally, the probability of tumor extinction is predicted for a fixed point in time. Alternatively or additionally, the probability of tumor extension is optionally predicted over a range of time.

In some implementations, the quantitative prediction of the cancer patient's response to the immune-based or targeted therapy is a progression-free survival (PFS).

Alternatively or additionally, the pre-treatment patient data is derived from a blood or tissue sample obtained at a time of or before administration of the immune-based or targeted therapy to the cancer patient.

Alternatively or additionally, the post-treatment patient data is derived from a blood or tissue sample obtained at a time after administration of the immune-based or targeted therapy to the cancer patient.

Optionally, the engineered cells are chimeric antigen receptor (CAR) T cells. Additionally, in these implementations, each of the pre-treatment patient data and the post-treatment patient data includes a measure of at least one of tumor volume, total lymphocytes, memory T cells, memory CAR T cells, effector CAR T cells, or antigen-presenting tumor cells.

In one aspect, an example treatment method is described. The method includes receiving pre-treatment patient data for a cancer patient, administering an immune-based or targeted therapy to the cancer patient, and receiving post-treatment patient data for the cancer patient. Each of the pre-treatment patient data and the post-treatment patient data includes a measure of at least one of tumor volume, total lymphocytes, memory T cells, memory engineered cells, tumor killing cells, or antigen-presenting tumor cells. The method also includes quantitatively predicting the cancer patient's response to the immune-based or targeted therapy using a model, the pre-treatment patient data, and the post-treatment patient data. The model is configured to represent dynamics and interactions among normal T cells, engineered cells, and tumor cells, and where the model includes a plurality of cell population compartments. The method further includes adjusting the immune-based or targeted therapy based upon the quantitative prediction, and administering the adjusted immune-based or targeted therapy to the cancer patient.

Optionally, the engineered cells are chimeric antigen receptor (CAR) T cells. Additionally, in these implementations, each of the pre-treatment patient data and the post-treatment patient data includes a measure of at least one of tumor volume, total lymphocytes, memory T cells, memory CAR T cells, effector CAR T cells, or antigen-presenting tumor cells.

In one aspect, an example system is described. The system includes a processor and a memory operably coupled to the processor, the memory having computer-executable instructions stored thereon. The system is configured to generate a model configured to represent dynamics and interactions among normal T cells, engineered cells, and tumor cells, where the model includes a plurality of cell population compartments. The system is also configured to receive pre-treatment patient data for a cancer patient, and receive post-treatment patient data for the cancer patient. Each of the pre-treatment patient data and the post-treatment patient data includes a measure of at least one of tumor volume, total lymphocytes, memory T cells, memory engineered cells, tumor killing cells, or antigen-presenting tumor cells. The system is further configured to quantitatively predict the cancer patient's response to an immune-based or targeted therapy using the model, the pre-treatment patient data, and the post-treatment patient data.

Optionally, the engineered cells are chimeric antigen receptor (CAR) T cells. Additionally, in these implementations, each of the pre-treatment patient data and the post-treatment patient data includes a measure of at least one of tumor volume, total lymphocytes, memory T cells, memory CAR T cells, effector CAR T cells, or antigen-presenting tumor cells.

In one aspect, an example computer-implemented method is described. The computer-implemented method includes receiving pre-treatment patient data for a cancer patient, and receiving post-treatment patient data for the cancer patient, where each of the pre-treatment patient data and the post-treatment patient data includes a measure of at least one of tumor volume, total lymphocytes, memory T cells, memory engineered cells, tumor killing cells, or antigen-presenting tumor cells. The computer-implemented method also includes quantitatively predicting the cancer patient's response to an immune-based or targeted therapy using a model, the pre-treatment patient data, and the post-treatment patient data. The model is configured to represent dynamics and interactions among normal T cells, engineered cells, and tumor cells, and where the model includes a plurality of cell population compartments.

Optionally, the engineered cells are chimeric antigen receptor (CAR) T cells. Additionally, in these implementations, each of the pre-treatment patient data and the post-treatment patient data includes a measure of at least one of tumor volume, total lymphocytes, memory T cells, memory CAR T cells, effector CAR T cells, or antigen-presenting tumor cells.

In one aspect, disclosed herein are methods for quantitatively predicting a cancer patient's response to immune-based or targeted therapy (such as, for example immunodepletion and CAR T cell infusion), comprising measuring primary patient data (such as for example measuring the tumor volume and/or measuring the number of cells from two or more cell populations selected from memory T cells, memory CAR T cells, effector CAR T cells, and antigen presenting tumor cells) for the cancer patient at time of administration of the immune-based or targeted therapy to create a baseline and measuring primary patient data following administration of the immune based or targeted therapy (such as, for example, at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 365 days; 1, 2, 3, 4, 5, 6, 7, 8, 9, 10, 11, 12, 13, 14, 15, 16, 17, 18, 19, 20, 21, 22, 23, 24, 25, 26, 28, 32, 36, 40, 44, 48, or 52 weeks; or 1, 2, 3, 4, 5, 6, 7, 8, 9, 10, 11, 12, 13, 14, 15, 16, 17, 18, 19, 20, 21, 22, 23, 24 months post administration of the immune-based or targeted therapy); wherein the primary patient data is derived from a blood or tissue sample; generating a plurality of secondary patient data (such as, for example, tumor growth rate, tumor cell extinction rate, memory T cell recovery rate, naïve/memory CAR T cell expansion rate, naïve/memory engineered cell differentiation rate, effector CAR T cell death rate, and/or effector CAR T cell exhaustion rate); and statistically analyzing the primary and secondary patient data to predict the cancer patient's response to immune-based or targeted therapy. In one aspect, the primary patient data (such as for example the cell populations) is measured at one or more days following administration of an immune-based targeted therapy.

Also disclosed herein are methods of treating, preventing, inhibiting, and/or reducing a cancer in a subject comprising administering to the subject comprising administering to the subject an immune-based or targeted therapy (such as, for example immunodepletion and CAR T cell infusion (including, but not limited to autologous CAR T cell infusion); measuring primary patient data (such as for example measuring the tumor volume and/or measuring the number of cells from two or more cell populations selected from memory T cells, memory CAR T cells, effector CAR T cells, and antigen presenting tumor cells) for the cancer patient, wherein the primary patient data is derived from a blood or tissue sample; generating a plurality of secondary patient data (such as, for example, tumor growth rate, tumor cell extinction rate, memory T cell recovery rate, naïve/memory CAR T cell expansion rate, naïve/memory engineered cell differentiation rate, effector CAR T cell death rate, and/or effector CAR T cell exhaustion rate); and statistically analyzing the primary and secondary patient data to detect the cancer patient's response to immune-based or targeted therapy; wherein a second round of immunodepleting therapy and an autologous CAR T cell infusion is administered by day 50 post autologous CAR T cell infusion when the tumor is still present 30 days after administration of the autologous CAR T cell infusion and wherein the initial tumor volume was less than 2.64×10¹¹ tumor cells and the tumor growth rate is less than 0.225/day or wherein the initial tumor volume was less than 1.05×10¹¹ tumor cells and the tumor growth rate is less than 0.25/day.

In one aspect, disclosed herein are methods of treating, preventing, reducing, and/or inhibiting a cancer in a subject of any preceding aspect, wherein the primary patient data (such as for example the cell populations) is measured at one or more timepoints (such as, for example, at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 365 days; 1, 2, 3, 4, 5, 6, 7, 8, 9, 10, 11, 12, 13, 14, 15, 16, 17, 18, 19, 20, 21, 22, 23, 24, 25, 26, 28, 32, 36, 40, 44, 48, or 52 weeks; or 1, 2, 3, 4, 5, 6, 7, 8, 9, 10, 11, 12, 13, 14, 15, 16, 17, 18, 19, 20, 21, 22, 23, 24 months post administration of the immune-based or targeted therapy) following administration of an immune-based targeted therapy.

Also disclosed herein are methods of treating, preventing, reducing, and/or inhibiting a cancer in a subject of any preceding aspect, wherein a second round of immunodepleting therapy and an autologous CAR T cell infusion is administered as soon as toxicity parameters allow where the initial tumor size is greater than 2.64×10¹¹ tumor cells and/or the tumor growth rate is faster than 0.25/day.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A-1F illustrate T cell interactions, CAR T cell compartmentalization, and tumor feedback on CAR T cell differentiation. FIG. 1A: Model schematic, assuming four cell compartments: memory (CCR7+) CAR T cells, M, proliferate and engage with resident lymphocytes, N (depleted by lymphodepleting chemotherapy), and differentiate into effector CAR T cells (CCR7−). E cells, with a finite life span, engage in killing CD19+ tumor and other B cells. Their production is impacted by CD19. FIG. 1B: On the level of individual cells, this system results in six cellular kinetic reactions. FIG. 1C: Schematic of data integration to parametrize the mathematical model; we used longitudinal data of peripheral absolute lymphocyte count (ALC), peripheral CAR+ cell counts per μL, and the tumor size changes as estimated from patients of the ZUMA-1 trial with complete response (CR) or progressive disease (PD). We assumed that, at days 30, 60, or 90, CRs had no detectable tumor mass, and that PDs had twice their initial tumor mass, Median initial tumor mass was 200 cm³. We seek to explain the dynamics of no response to CAR therapy (FIG. 1D), transient response followed by progression/relapse (FIG. 1E), and long term/complete response (tumor is eradicated) (FIG. 1F). These example dynamics were generated using Equations (M1)-(M4) with hand-picked parameters.

FIGS. 2A-2D illustrate a population-level model of T cell co-evolution, complex CAR T cell dynamics can predict clinical endpoints as stochastic events. FIG. 2A: ALC was used to parameterize normal T cell dynamics, Eq. (M1). Estimation did not change dramatically based on ALC or ALC-CAR, more time points were available for ALC. FIG. 2B: CAR positive T cell dynamics (Eqs. (M2), (M3)) were parameterized using ZUMA-1 trial data of median peripheral CAR counts, to fit peak and decay of CAR. The nonlinear optimization for data fitting (Example 2) revealed multiple trajectories that fit the data equally well, of which several are shown. FIG. 2C: Memory CAR T cell expansion rate decreases over time by up to three orders of magnitude, possibly as a result of immunogenicity. Violin plot insets: distributions of CAR memory expansion parameters for different realizations of the fitting procedure (Methods, Example 2). FIG. 2D: Two example trajectories of tumor burden over time, using identical parameters and initial conditions for the stochastic process shown in FIG. 1 B. Both examples enter the stochastic region (<100 tumor cells), but one escapes this extinction vortex leading to progression. All parameter values and initial conditions used are given in Table 1. Stochastic simulation procedure described in the Example 2.

FIG. 3A-3G illustrate leveraging the stochastic simulation framework to describe progression-free survival and probability of cure. FIG. 3A: Increasing the fraction of initial memory CAR T cells (e.g. marked as CCR7+) can improve chances of cure. FIG. 3B: Initial ALC of six cells/4 at CAR administration due to lymphodepletion is crucial; increasing this number would monotonously decrease the chances of cure. FIG. 3C: Progression-free survival (PFS) was recorded in ZUMA-1 (gray line). Our stochastic model, parameterized using normal and CAR T cell counts together with tumor status at days 30, 60, 90, recapitulates this PFS curve, using simulations with a mixture of parameters drawn from a normal distribution with a variance of 15% of the mean (Methods, Example 2). We recorded progression when 2 times the initial tumor mass was reached to avoid possible bias against cases that briefly increased in tumor mass but responded well after day 5, which changes survival curves minimally (see discussion in the Example 2). Intrinsic tumor growth rates: 0.115/day (slow), 0.19/day (medium), 0.265/day (fast). FIG. 3D, FIG. 3E: Fine-grained comparison of survival at distinct time points, as a function of initial tumor burden (FIG. 3D) and tumor growth rate (FIG. 3E). FIG. 3F: The distribution of cure times for the median parameters. Most patients are cured before day 100. FIG. 3G: The distribution of progression times for the median parameters. Most patients progress between days 80-400. All parameter values used are given in Table 1. All probabilities estimated used 1000 stochastic simulations with the same initial conditions.

FIGS. 4A-4C illustrate timing of a switch to low CAR T cell fitness could determine feasibility and magnitude of a second CAR T cell dose. FIG. 4A: Recall that we found that memory CAR T cells transition to a lower intrinsic growth rate around day 19 (solid line). One could, however, imagine that this effect could be delayed with suitable intervention (dashed line). FIG. 4B: In the original scenario, in concordance with observations by Turtle et al.¹, second dose of CAR T cells at a reasonable time point (past day 20) has no effect in raising long-term PFS (measured as day 700 post first dose). FIG. 4C: in contrast, should it be possible to delay the loss of initial CAR T cell fitness, a second dose could be given later to result in a noticeable increase in survival. Except for the indicated intrinsic tumor growth rates, all parameter values used are given in Table 1. All probability estimated used 1000 stochastic simulations with the same initial conditions and a hyperparameter of 15% parameter variance. PFS was evaluated at day 700.

FIG. 5A: Quartile data of CAR T cell count in periphery reproduced from 101 patients of the ZUMA-1 trial [5] (lower quartile: circles, median: squares, upper quartile: discs). Of note, the initial median CAR T cell dose (at time 0) was 0.36 cell per μL blood—the fitting procedure presented here only accounts for the decay of CAR and cannot describe its initial increase. FIG. 5B: We fit exponential and power law decay to the data (using LinearModelFit in Wolfram Mathematica), resulting in different values of the respective decay parameter 6. Throughout, power law decay shows a significantly improved fit to these data points. FIG. 5C: Exponential (exp^{-ßt}, dashed) and power law (t^{-ß}, solid) fits to the lower quartile data (circles) on the top, respective fit residuals on the bottom. FIG. 5D: Exponential (exp^{-ßt}, dashed) and power law (t^{-ß}, solid) fits to the median data (squares) on the top, respective fit residuals on the bottom. FIG. 5E: Exponential (exp^{-ß}, dashed) and power law (t^{-ß}, solid) fits to the upper quartile data (disks) on the top, respective fit residuals on the bottom.

FIG. 6 illustrates a comparison of rM (t)=rM (pink) and rM as defined in (20) (blue), showing the improvement gained by introducing a growth rate switch.

FIGS. 7A-7B are box plots showing the ranges of parameter values obtained from the fitting routine.

FIG. 8A: Progression defined by the clinical cutoff of a 50% increase (ß=1.5). High growth rate tumors cause the simulation to classify early progression on these fast tumors. FIG. 8B: With medium to low growth rate tumors, all cutoffs lead to similar PFS curves, with the shifts being explained by the additional time to reach that size (eq. (39)). The small shift for long-term PFS is within the expected deviation of a 10 k patient sample, which has a maximum 95% CI of p*±0.01 where p* is long-term PFS. In contrast, FIG. 8C: highlights the potential pitfalls of smaller cutoffs for the simulation, which result in early progression. In fact, some of these progressions would ultimately have been classified as cured, as the purple shows (with ß=1.5, and early progressions removed).

FIGS. 9A-9E show how the mathematical model recapitulates and predicts progression-free survival (PFS), and can suggest actionable therapy improvements. 1000 simulated patients were used to generate each PFS curve. FIG. 9 A: Impact of parameter variation (hyperparameter σ) on the PFS. All subsequent panels use σ=0.15. FIG. 9 B: Impact of tumor growth rate on the PFS. FIG. 9 C: Impact of initial tumor size on the PFS. Much larger tumors lead to some patients progressing earlier since the CAR could not expand fast enough. FIG. 9 D: Impact of CAR T infusion phenotype composition. In general, a higher memory fraction lead to better PFS rates. FIG. 9 E: Impact of lymphodepletion on PFS. Similar to FIG. 2G (Main text), a sizable impact on PFS is observed by doubling or tripling the amount of normal T cells after depletion. All parameter values used are given in Table 1. All probability estimated used 1000 stochastic simulations with the same initial conditions.

FIG. 10 is an example computing device.

FIG. 11 includes Table 1, which shows the median parameter values of our model, using minimization of the loss functions (19) and (22). Model fitting used T cell densities in cells per μL (peripheral) and tumor size in cubic cm. Stochastic hybrid simulation modeling used transformation to cell counts, assuming that every patient has on average 5 L of blood that contains 1% of the T or CAR T cell population, and that 1 cm³ contains 10⁹ tumor cells on average. Initial conditions are the median values. ALC: absolute lymphocyte count.

FIG. 12 is a flowchart illustrating example operations for quantitatively predicting a cancer patient's response to an immune-based or targeted therapy according to an implementation described herein.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Methods of Treating Cancer

Large B cell Lymphoma (LBCL) is curable in a majority of patients treated with upfront chemoimmunotherapy. Historically, the prognosis for patients refractory to first or second line therapy is dismal, with only 7% able to achieve a complete response to the next line of chemotherapy and a median overall survival of less than 7 months. These patients can now benefit from Chimeric Antigen Receptor (CAR) T cell therapies, such as axicabtagene ciloleucel (axi-cel) which consists of autologous engineered T cells re-targeted against the CD19+ lymphoma. Several factors are known to be associated with efficacy of CAR T cell therapy, including the size of lymphoma tumor mass, the degree of T cell expansion, the number CAR T cells with a less differentiated phenotype within the infusion product, and the degree of lymphodepletion provided by conditioning chemotherapy prior to CAR T infusion. What remains largely undescribed is a mechanistic understanding of how these individual processes relate to each other and to the desired outcome of long term durable remission following CAR T therapy. The relationships of these factors over time can be fundamentally described by using patient data, statistical data analyses, and mathematical modeling. This mathematical model sheds light on the biological laws underlying CAR T cell expansion, wildtype T cell repopulation, and tumor eradication after induction chemotherapy and CAR infusion. Furthermore this model can be used for in silico testing of strategies to improve CAR T therapy outcomes, such as the impact and timing of additional infusions. Alternatively it can be integrated into clinical trial design as a tool to understand why experimental interventions did or did not increase the efficacy of CAR T therapy.

Disclosed herein are methods of treating, preventing, inhibiting, and/or reducing a cancer in a subject comprising administering to the subject comprising administering to the subject an immune-based or targeted therapy (such as, for example immunodepletion and CAR T cell infusion (including, but not limited to autologous CAR T cell infusion); measuring primary patient data (such as for example measuring the tumor volume and/or measuring the number of cells from two or more cell populations selected from memory T cells, memory CAR T cells, effector CAR T cells, and antigen presenting tumor cells) for the cancer patient, wherein the primary patient data is derived from a blood or tissue sample; generating a plurality of secondary patient data (such as, for example, tumor growth rate, tumor cell extinction rate, memory T cell recovery rate, naïve/memory CAR T cell expansion rate, naïve/memory engineered cell differentiation rate, effector CAR T cell death rate, and/or effector CAR T cell exhaustion rate); and statistically analyzing the primary and secondary patient data to detect the cancer patient's response to immune-based or targeted therapy; wherein a second round of immunodepleting therapy and an autologous CAR T cell infusion is administered by day 50 post autologous CAR T cell infusion when the tumor is still present 30 days after administration of the autologous CAR T cell infusion and wherein the initial tumor volume was less than 2.64×10¹¹ tumor cells and the tumor growth rate is less than 0.225/day or wherein the initial tumor volume was less than 1.05×10¹¹ tumor cells and the tumor growth rate is less than 0.25/day. Where the tumor initial tumor size is greater than 2.64×10¹¹ tumor cells and/or the tumor growth rate is faster than 0.25/day a second round of immunodepleting therapy and an autologous CAR T cell infusion can be administered as soon as toxicity parameters allow.

It is understood and herein contemplated that the immune-based or targeted therapy can include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate) as well as not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). In one aspect, the immune based or targeted therapy comprises immunodepletion followed by administration of a CAR T cells (i.e., CAR T cell infusion). The CAR T cells can come from any immunocomatible source including, but not limited to autologous CAR T cells. The CAR T cells can be designed to target any tumor antigen known in the art including, but not limited to a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, ß-human chorionic gonadotropin, alphafetoprotein (AFP), ALK, CD19, CD123, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin.

In one aspect, the disclosed herein are methods of treating, preventing, reducing, and/or inhibiting a cancer in a subject comprise obtaining primary patient data at multiple timepoints. In one aspect the primary patient data (such as for example the cell populations) is measured at one or more timepoints (such as, for example, at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 365 days; 1, 2, 3, 4, 5, 6, 7, 8, 9, 10, 11, 12, 13, 14, 15, 16, 17, 18, 19, 20, 21, 22, 23, 24, 25, 26, 28, 32, 36, 40, 44, 48, or 52 weeks; or 1, 2, 3, 4, 5, 6, 7, 8, 9, 10, 11, 12, 13, 14, 15, 16, 17, 18, 19, 20, 21, 22, 23, 24 months post administration of the immune-based or targeted therapy) following administration of an immune-based targeted therapy.

The disclosed compositions can be used to treat any disease where uncontrolled cellular proliferation occurs such as cancers. A non-limiting list of different types of cancers that the disclosed methods can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Example Methods

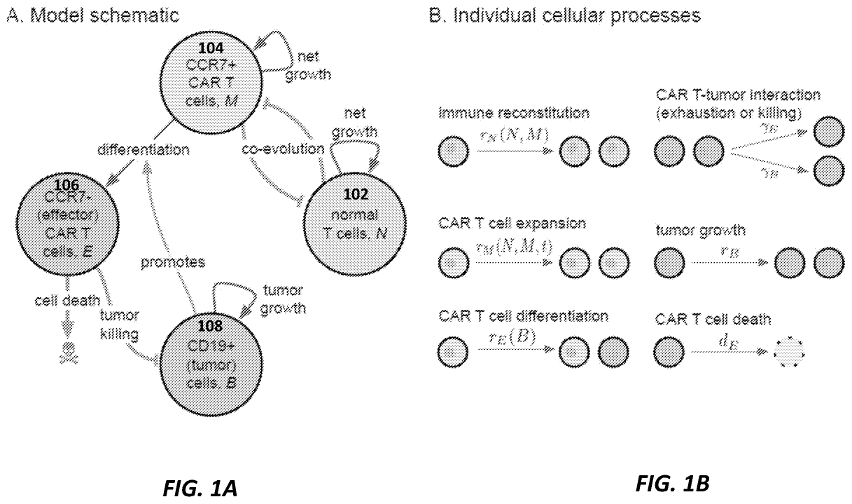

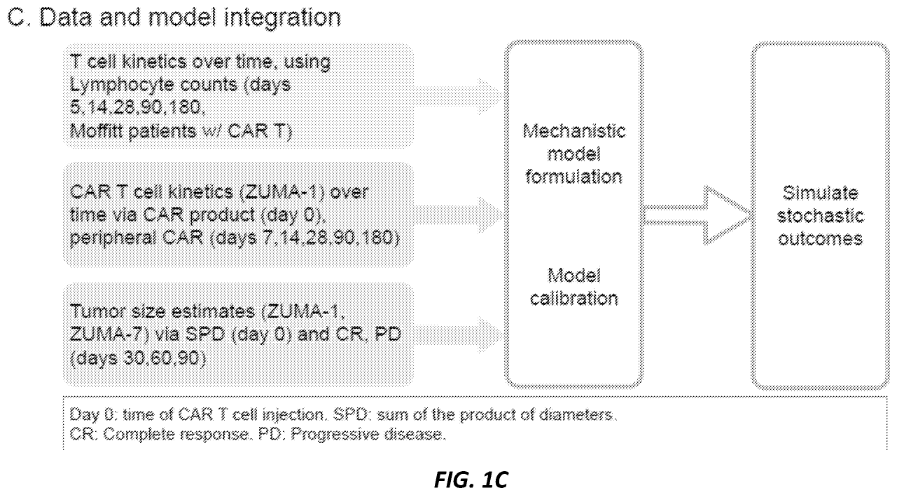

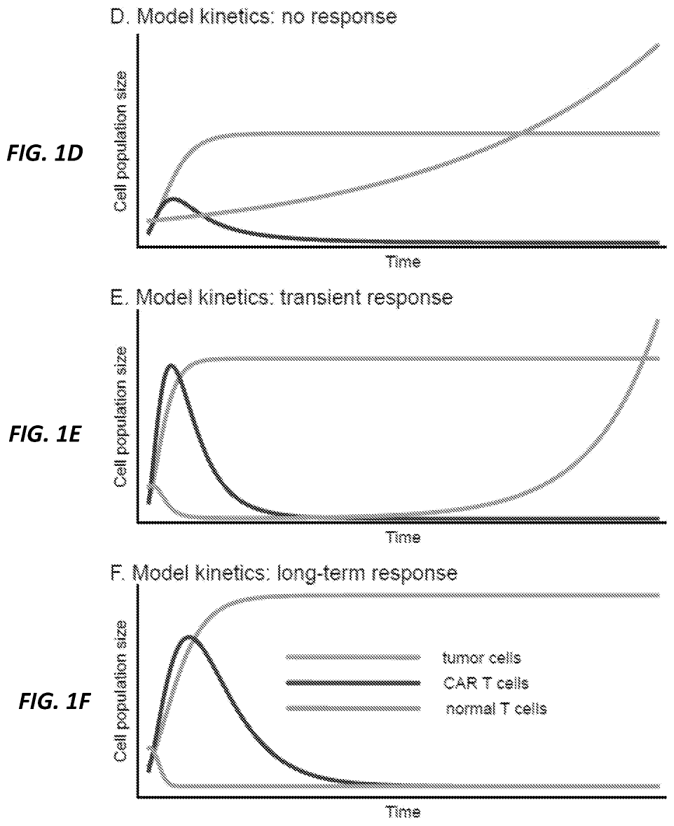

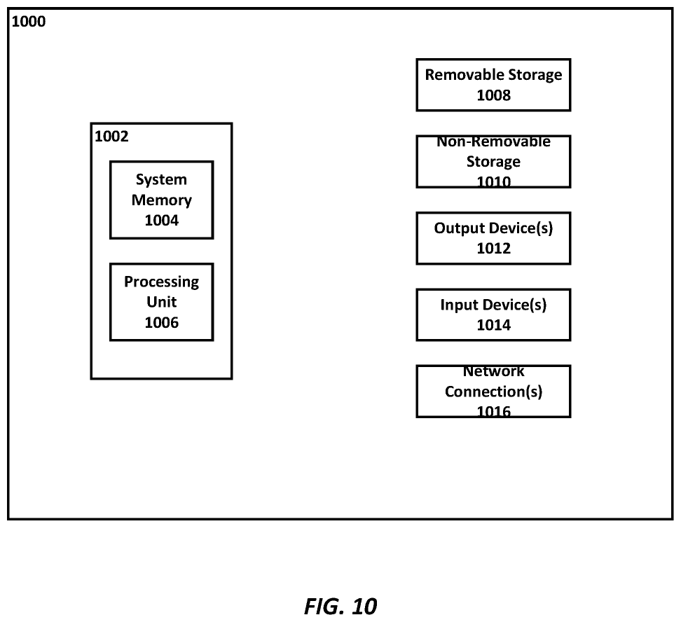

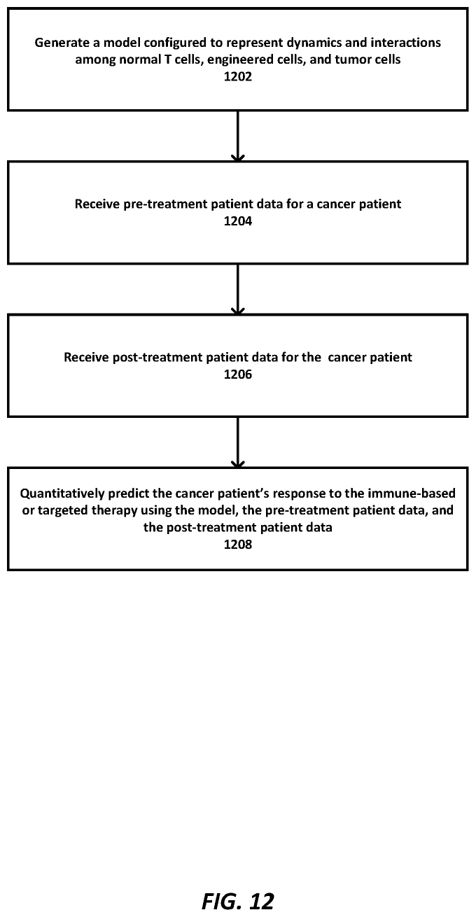

Referring now to FIG. 12, example operations for quantitatively predicting a cancer patient's response to an immune-based or targeted therapy are shown. This disclosure contemplates that the operations shown in FIG. 12 can be performed using a computing device (e.g., computing device 1000 of FIG. 10). As described herein, tumor eradication in response to immune-based or targeted therapy is a stochastic event, which, even when likely, can occur at variable times. In other words, while it is possible to statistically analyze the likelihood of tumor eradication, the event is still difficult to predict. The method described with regard to FIG. 12 can be used to address difficulties in predicting patient response to immune-based or targeted therapy. In particular, the complex extinction process of the model described herein (see step 1202, FIGS. 1A and 1B) is leveraged to quantitatively predict patient response.

At step 1202, a model configured to represent dynamics and interactions among normal T cells, engineered cells, and tumor cells is generated. The model includes a plurality of cell population compartments (e.g., normal naïve/memory T cells, naïve/memory engineered cells, tumor killing cells, and antigen-presenting tumor cells). In these implementations, the plurality of cell population compartments can be modelled based on continuous-time birth and death stochastic processes and deterministic mean-field equations. The model is configured to simulate interactions between normal T cells and engineered cells. Alternatively or additionally, the model is configured to simulate a differentiation rate of memory engineered cells to tumor killing cells. An example model is shown in FIGS. 1A and 1B. For example, in some implementations, the engineered cells are chimeric antigen receptor (CAR) T cells. Example 1 and 2 below describe implementations where the engineered cells are CAR T cells. It should be understood that CAR T cells are provided only as an example. This disclosure contemplates that the engineered cells can be other types of engineered cells including, but not limited to, CAR natural killer cells (CAR NK cells), CAR B cells, tumor-infiltrating lymphocytes (TILs), etc.

At step 1204, pre-treatment patient data for a cancer patient is received. It should be understood that pre-treatment patient data is collected at a time of or before administration of the immune-based or targeted therapy to the cancer patient. This disclosure contemplates that the patient data can be stored, for example, in memory of a computing device. Patient data includes a measure of at least one of tumor volume, total lymphocytes, memory T cells, memory engineered cells (e.g., memory CAR T cells), tumor killing cells (e.g., effector CAR T cells), or antigen-presenting tumor cells. In some implementations, patient data includes a plurality of measures, e.g., two or more of tumor volume, total lymphocytes, memory T cells, memory engineered cells (e.g., memory CAR T cells), tumor killing cells (e.g., effector CAR T cells), or antigen-presenting tumor cells. It should be understood that one or more of the measures may be derived from a blood or tissue sample taken from the cancer patient. This disclosure contemplates that the blood or tissue sample can be analyzed using any techniques known in the art, for example blood test (e.g., B and T cell screen), flow cytometry, or other technique. Alternatively or additionally, it should be understood that one or more of the measures can be based on the therapy dosage (e.g., the initial memory CAR T cells). In some implementations, the pre-treatment patient data may include tumor volume, absolute lymphocyte count (ALC), and memory CAR T cells. Alternatively or additionally, this disclosure contemplates that the patient data may include other data including, but not limited to, the patient's age and/or metabolic fitness.

At step 1206, post-treatment patient data for the cancer patient is received. It should be understood that post-treatment patient data is collected at a time after administration of the immune-based or targeted therapy to the cancer patient. This disclosure contemplates that the patient data can be stored, for example, in memory of a computing device. As described above, patient data includes a measure of at least one of tumor volume, total lymphocytes, memory T cells, memory engineered cells (e.g., memory CAR T cells), tumor killing cells (e.g., effector CAR T cells), or antigen-presenting tumor cells. In some implementations, patient data includes a plurality of measures, e.g., two or more of tumor volume, total lymphocytes, memory T cells, memory engineered cells (e.g., memory CAR T cells), tumor killing cells (e.g., effector CAR T cells), or antigen-presenting tumor cells. Alternatively or additionally, the post-treatment patient data optionally further includes a measure of at least one tumor growth rate, tumor cell extinction rate, memory T cell recovery rate, naïve/memory engineered cell (e.g., memory CAR T cell) expansion rate, naïve/memory engineered cell differentiation rate, tumor killing cell (e.g., effector CAR T cell) death rate, or tumor killing cell exhaustion rate. In some implementations, the post-treatment patient data may include tumor volume, absolute lymphocyte count (ALC), memory CAR T cells, and effector CAR T cells. Alternatively or additionally, this disclosure contemplates that the patient data may include other data including, but not limited to, the patient's age and/or metabolic fitness.

At 1208, the cancer patient's response to the immune-based or targeted therapy is quantitatively predicted using the model, the pre-treatment patient data, and the post-treatment patient data. In some implementations, the quantitative prediction is a probability of tumor extinction. Optionally, the probability of tumor extinction is predicted for a fixed point in time. Alternatively or additionally, the probability of tumor extension is optionally predicted over a range of time (e.g., integrated over time). In some implementations, the quantitative prediction is a progression-free survival (PFS).

A method for quantitatively predicting a cancer patient's response to an immune-based or targeted therapy according to another implementation is described below. This disclosure contemplates that this method can be performed using a computing device (e.g., computing device 1000 of FIG. 10). The method can include receiving pre-treatment patient data for a cancer patient, and receiving post-treatment patient data for the cancer patient. Pre- and post-treatment patient data are described above in detail with regard to steps 1204 and 1206 of FIG. 12. The method can also include quantitatively predicting the cancer patient's response to an immune-based or targeted therapy using a model, the pre-treatment patient data, and the post-treatment patient data. The model is described above in detail with regard to step 1202. Similarly to FIG. 12, in some implementations, the quantitative prediction is a probability of tumor extinction. Optionally, the probability of tumor extinction is predicted for a fixed point in time. Alternatively or additionally, the probability of tumor extension is optionally predicted over a range of time.

A method of treatment is also described below. The method includes receiving pre-treatment patient data for a cancer patient (see step 1204, FIG. 12), administering an immune-based or targeted therapy to the cancer patient, and receiving post-treatment patient data for the cancer patient (see step 1206, FIG. 12). The method also includes quantitatively predicting the cancer patient's response to the immune-based or targeted therapy using a model, the pre-treatment patient data, and the post-treatment patient data (see step 1208, FIG. 12). The model is described above in detail with regard to step 1202. The method further includes adjusting the immune-based or targeted therapy based upon the quantitative prediction, and administering the adjusted immune-based or targeted therapy to the cancer patient.

Example Computing System

It should be appreciated that the logical operations described herein with respect to the various figures may be implemented (1) as a sequence of computer implemented acts or program modules (i.e., software) running on a computing device (e.g., the computing device described in FIG. 10), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 10, an example computing device 1000 upon which the methods described herein may be implemented is illustrated. It should be understood that the example computing device 1000 is only one example of a suitable computing environment upon which the methods described herein may be implemented. Optionally, the computing device 1000 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 1000 typically includes at least one processing unit 1006 and system memory 1004. Depending on the exact configuration and type of computing device, system memory 1004 may be volatile (such as random access memory (RAM)), non-volatile (such as read-only memory (ROM), flash memory, etc.), or some combination of the two. This most basic configuration is illustrated in FIG. 10 by dashed line 1002. The processing unit 1006 may be a standard programmable processor that performs arithmetic and logic operations necessary for operation of the computing device 1000. The computing device 1000 may also include a bus or other communication mechanism for communicating information among various components of the computing device 1000.

Computing device 1000 may have additional features/functionality. For example, computing device 1000 may include additional storage such as removable storage 1008 and non-removable storage 1010 including, but not limited to, magnetic or optical disks or tapes. Computing device 1000 may also contain network connection(s) 1016 that allow the device to communicate with other devices. Computing device 1000 may also have input device(s) 1014 such as a keyboard, mouse, touch screen, etc. Output device(s) 1012 such as a display, speakers, printer, etc. may also be included. The additional devices may be connected to the bus in order to facilitate communication of data among the components of the computing device 1000. All these devices are well known in the art and need not be discussed at length here.

The processing unit 1006 may be configured to execute program code encoded in tangible, computer-readable media. Tangible, computer-readable media refers to any media that is capable of providing data that causes the computing device 1000 (i.e., a machine) to operate in a particular fashion. Various computer-readable media may be utilized to provide instructions to the processing unit 1006 for execution. Example tangible, computer-readable media may include, but is not limited to, volatile media, non-volatile media, removable media and non-removable media implemented in any method or technology for storage of information such as computer readable instructions, data structures, program modules or other data. System memory 1004, removable storage 1008, and non-removable storage 1010 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1006 may execute program code stored in the system memory 1004. For example, the bus may carry data to the system memory 1004, from which the processing unit 1006 receives and executes instructions. The data received by the system memory 1004 may optionally be stored on the removable storage 1008 or the non-removable storage 1010 before or after execution by the processing unit 1006.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Chimeric antigen receptor (CAR) T cell therapy is a remarkably effective immunotherapy that relies on in vivo expansion of engineered CAR T cells, after lymphodepletion by chemotherapy. The laws underlying this expansion and subsequent tumor eradication remain unknown. Here we seek to disentangle the processes that contribute to CAR expansion and tumor eradication. We develop a mathematical model of T cell-tumor cell interactions, and demonstrate that CAR expansion is shaped by immune reconstitution dynamics after lymphodepletion and predator prey-like dynamics among T cells. Therapy is effective because CAR T effector cells rapidly grow and engage the tumor, but CAR T cells experience an emerging growth rate-disadvantage, possibly due to immunogenicity. We parameterized the model using patient population-level CAR and tumor data over time and recapitulate progression-free survival rates independently. We find that tumor eradication is a stochastic event, which, even when likely, can occur at variable times. While possible cure events caused by this extinction vortex occur early and are narrowly distributed, progression events occur late and are widely distributed in time. Our approach highlights the roles of complex interactions and stochastic effects, and is the first eco-evolutionary dynamics approach to clinical data of CAR T cell therapy. Our complex extinction process can be leveraged to further quantify why therapy works in some patients but not others, to understand immunogenicity, T cell exhaustion, and possibly toxicity of the cellular therapy.

Introduction

Relapsed and refractory large B cell lymphoma (LBCL) is the most common subtype of Non-Hodgkin Lymphoma, which itself was the most common hematologic malignancy in the US with 72,000 new cases (4.3% of all cancer) and 20,000 deaths (3.4% of all cancer deaths) in 2017². In LBCL patients that do not respond to chemotherapy, the median overall survival is under 7 months³. These patients could benefit from autologous chimeric antigen receptor (CAR) T cell therapy that uses genetically engineered T cells re-targeted to CD19, a protein specific to B lineage cells from which LBCL arises⁴. ZUMA-1 was a pivotal, multi-center, phase 1-2 trial of axicabtagene ciloleucel (axi-cel, n=101 patients treated)⁵⁻⁷. Overall Response Rate (ORR) and Complete Response (CR) rate were 82% and 54%—respective responses would have been 26% and 7% with standard chemotherapy³. While many LBCL patients see a temporary reduction in tumor burden, about 60% eventually still progress.

Quantitative models of CAR T dynamics and tumor responses are needed to better predict patient outcomes such as cure and progression. Cellular immunotherapies, such as CAR T cell therapy, describe a new frontier for predictive mathematical biological modeling⁸⁻¹¹. Recent works used mixed-effect modeling of CAR-T cell therapy⁸ to model the expansion of the CAR T cell drug tisagenlecleucel¹², in combination with therapies that treat cytokine release syndrome. Interestingly, this model assumed a CAR T cell compartmentalization as an explanatory approach to the complex CAR dynamics over time. Others considered eco-evolutionary dynamics to explain CAR T cell expansion and exhaustion¹⁰, and signaling-induced cell state variability¹¹, both inferred from in vitro data and without considering interactions between T cells and tumor. Here, we seek a fundamental understanding of T and CAR T cells including tumor cell dynamics in vivo using mathematical modeling.

We analyzed and integrated clinical data of T and CAR T expansion and tumor response during CAR T cell therapy, in order to discover the mathematical principles that drive durable response or relapse and progression. We focus on the two CAR T cell differentiation states of naïve/long-term memory and effector cells^{13,14}, and propose that co-evolution in the T cell homeostatic niche drives the observed CAR T cell dynamics, whereby there is feedback of tumor antigen on CAR T cell differentiation¹⁵. An improved understanding of these mechanistic interactions and dynamics can be leveraged to better understand the dynamics of progression, and explore new personalized dosing and combination therapies.

Methods

We developed a mathematical modeling framework that describes dynamics and interactions among normal T cells, CAR T cells, and tumor cells. The model considers four cell populations in the form of continuous-time birth and death stochastic processes and their deterministic mean-field equations: normal naïve/memory T cells, N, naïve/memory CAR T cells, M, effector CAR T cells, E, and antigen-presenting tumor cells, B.

We consider the case of lymphodepleting chemotherapy prior to infusion of autologous CAR T cells. We set time to 0 at the time of CAR infusion. Normal and CAR memory T cell populations then grow toward their respective carrying capacities and influence each other. This mutual influence gives rise to co-evolution. Normal memory cells expand at the rate r_{N}*log[K_{N}/(N+M)]. CAR memory cells, for our purposes labeled as CCR7+^{13,15,16}, expand at the rate r_{M}*log[K_{M}/(N+M)]. Of note, we allow for a time-dependent intrinsic growth rate of memory CAR T cells around time

$$τ > 0 , {r}_{M} ( t ) = \frac{{r}_{M , max} - {r}_{M , min}}{1 + {e}^{t - τ}} + {r}_{M , min} .$$

This transition from a large to a smaller growth rate r_{M} can be attributed to acquired immunogenicity. Around a characteristic time, the wildtype T cells begin to mount an attack against the murine sequences present in the CAR T cells, effectively reducing CAR T expansion.

Memory CAR T cells differentiate into effector CAR T cells, and become CCR7-^{13,15,16}, at a rate r_{E}(B), depending on the amount of CD19 antigen present. In the model, we exclude possible influences by other sources of CD19, such as normal B cells. In ZUMA-1, ^{˜}50% of patients did not have detectable normal B cells in circulation at the time of lymphodepletion, and 3 months after CAR T infusion, less than 20% of patients had detectable normal B cells. Thus, in the patient population we are modeling, normal sources of CD19 do not seem to play a significant role during the time CAR T cells are most active. We used the following antigen-driven, piecewise-linear feedback of tumor mass on the rate at which effector CAR T cells emerge: r_{E}(B)=r_{E} (0)(1+α₁ min{B/B₀, α₂}).

Memory T cells have a very long life-span relative to the time scale of CAR expansion. Therefore, the death rate of the memory compartments are neglected. In theory, our model allows for CAR memory to persist, but only if CAR T cells have a long-term competitive advantage, Kₘ≥Kₙ, which is unlikely.

In contrast, effector CAR T cells have a maximum life-span influenced by their intrinsic death rate d_{E} and the interaction with CD19+ tumor cells, which also leads to exhaustion and subsequent effector cell death at rate γ_{E}¹⁷.

The tumor cell population B grows autonomously at the net growth rate r_{B}, and experiences tumor killing at rate γ_{B}, proportional to the number of CAR T effector cells E. This dynamical system can be written, in the mean-field limit, as a set of deterministic ordinary differential equations:

$$\frac{dN}{dt} = - {r}_{N} N log [ \frac{N + M}{{K}_{N}} ] \qquad ( M1 )$$

$$\frac{dM}{dt} = - {r}_{M} ( t ) M log [ \frac{N + M}{{K}_{M}} ] \qquad ( M2 )$$

$$\frac{dE}{dt} = {r}_{E} ( B ) M - {γ}_{E} BE - {d}_{E} E \qquad ( M3 )$$

$$\frac{dB}{dt} = {r}_{B} B - {γ}_{B} BE \qquad ( M4 )$$

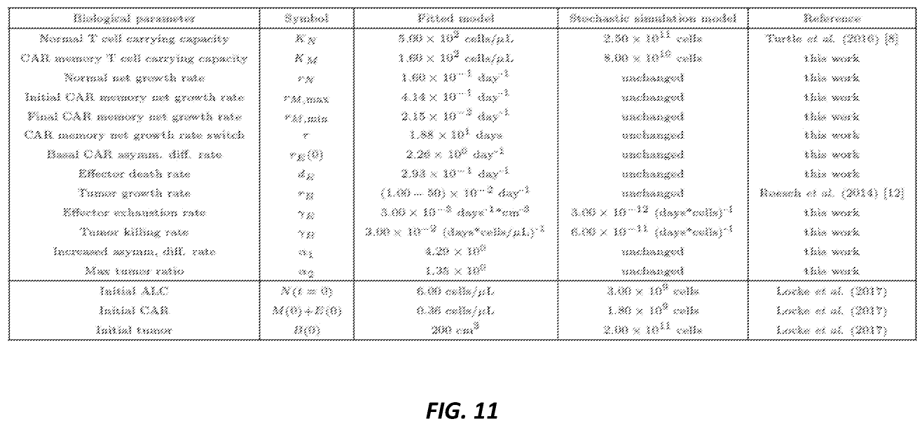

For a derivation of these equations and their associated stochastic birth and death process, see Example 2, where we also present a stability analysis. FIG. 1 A shows a schematic of this complex dynamical system, FIG. 1 B the cellular events. We calibrated the model using clinical data (see FIG. 1 C, Table 1 (FIG. 11), Example 2). The deterministic system's qualitative behavior aligns with clinical observations (FIG. 1 D-F). However, to recapitulate and predict progression-free survival over time, we used a corresponding a stochastic formulation that is able to capture dynamics of small tumor populations near extinction.

Results

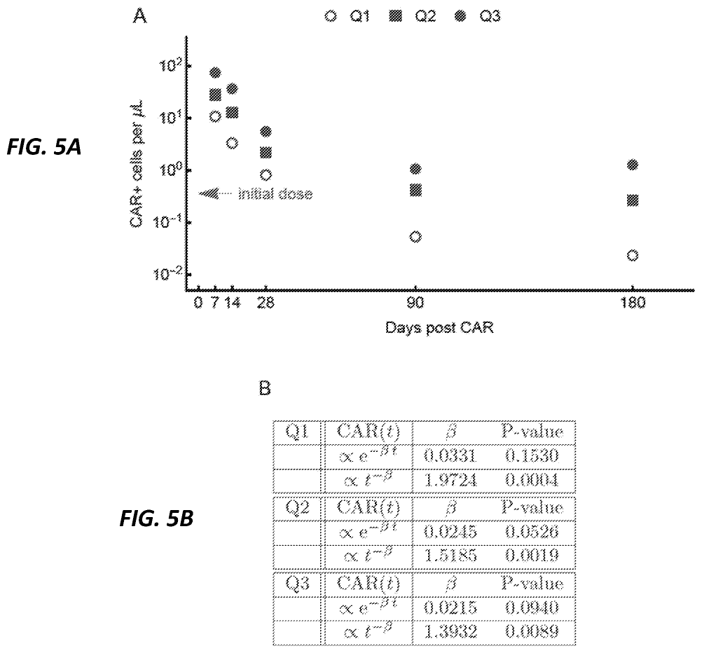

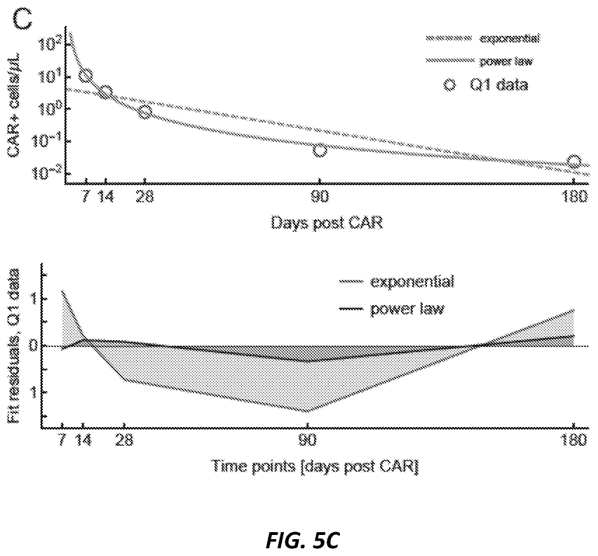

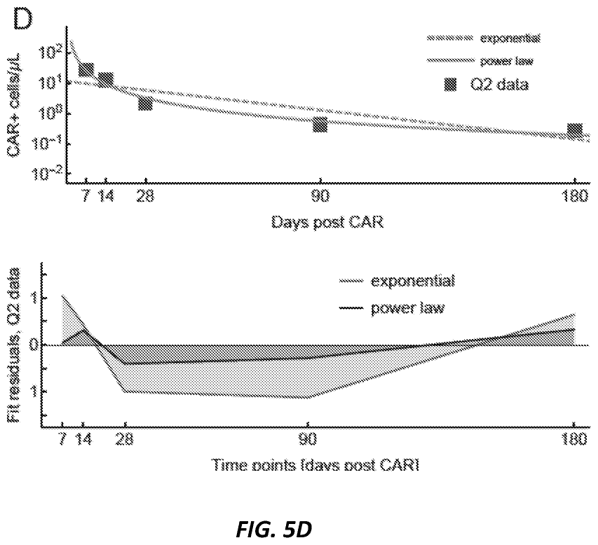

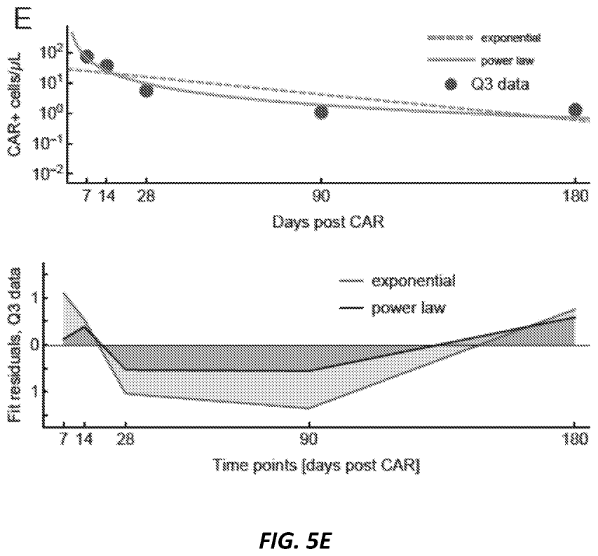

Cellular immunotherapies pose an interesting novel modeling challenge, as they are active, living treatments that proliferate and interact with other cells and signals of the immune system. These interactions lead to complex dynamical drug concentration profiles. The concentration of CAR T cells after administration does not follow exponential growth toward a peak followed by exponential decay. Rather, analysis of the longitudinal data indicates that multiple scales within the CAR T cell population are relevant, CAR T decay follows a prolonged, power law-like decay (FIG. 5A-D).

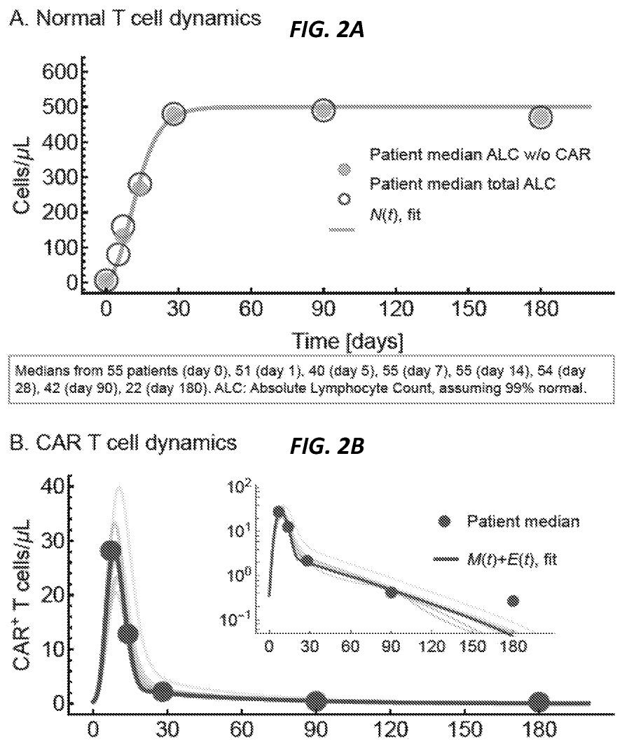

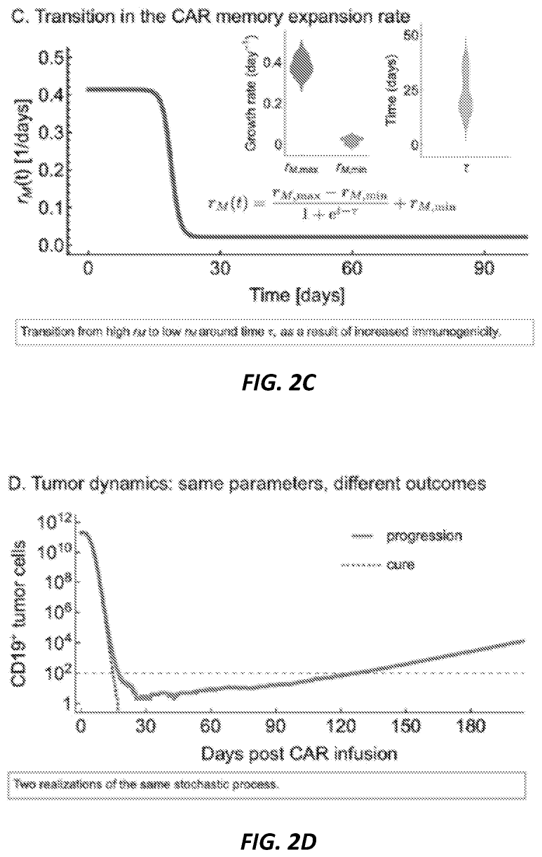

CAR T cell expansion has been associated with durable response to therapy^{5,8}. Two key components of our model are mechanisms that drive CAR T expansion and persistence: (a) external homeostatic signals (normal T-CAR T interactions), (b) CAR memory to effector differentiation rate, which is proportional to tumor mass. We fit our model to normal lymphocyte (FIG. 2 A) and CAR T cell dynamics (FIG. 2 B), resulting in a model calibration (Table 1).

Co-Evolution Among Normal T and CAR T Cells

We leveraged absolute lymphocyte count (ALC) data to quantify possible selection against CAR T cells (FIG. 2 A). We assumed that mutual inhibitory co-evolutionary dynamics occurred mostly on the level of memory cells. Memory CAR T cells experience an initial growth advantage, driven by an initial difference in growth rates of 0.16/day in normal vs. 0.41/day in CAR memory T cells. This initial growth advantage is overruled by an almost three-fold higher long-term carrying capacity of normal T cells (500¹⁸ vs. 160 cells per 4) leading to their characteristic non-monotonic trajectory (FIG. 2 B), accompanied by a mounting selection that further lowers CAR T cell expansion (FIG. 2 C). We estimate that the CAR T cell carrying capacity is reduced to about 33% of their normal T cell counterpart.

Time-Dependent Memory CAR Expansion Rate as a Result of Mounting Immunogenicity

CAR T cells are the result of complex ex vivo engineering, often based on non-human (murine) constructs¹⁹. We discovered a transition from rapid to slow growth around day 19 post CAR injection (FIG. 2 C). During this switch, the memory CAR T cells' expansion rate is reduced from 0.41/day, which gave them their initial competitive advantage, to about 0.02/day. Due to additional rapid differentiation into effector CAR T cells at a baseline rate of 2.26/day and proportional to tumor antigen the overall CAR signal exhibits a peak. Therapy success crucially depends on the CAR's ability to mount an effective anti-tumor response before memory cells lose their initial advantage, possibly as immunogenicity takes effect.

Stochastic Tumor Extinction can be an Explanation for Observed Treatment Success Rates

In realistic clinical settings CAR T cells are slightly or strongly maladapted in the long run. Tumor eradication is deterministically unstable and not a long-term outcome (see Example 2). However, tumor mass often shrinks at least for some time and can temporarily be brought down to very small values. To describe the observed rates of long-term tumor reduction, tumors of small size are severely impacted by extinction events (FIG. 2 D).

Malignant B cell extinction is a stochastic event driven by CAR T cell expansion and anti-tumor activity. Tumors can be driven close to an extinction vortex²⁰. This means that, even if cure is likely under clinically favorable conditions (and progression unlikely), the time to cure or progression can follow a broad distribution.

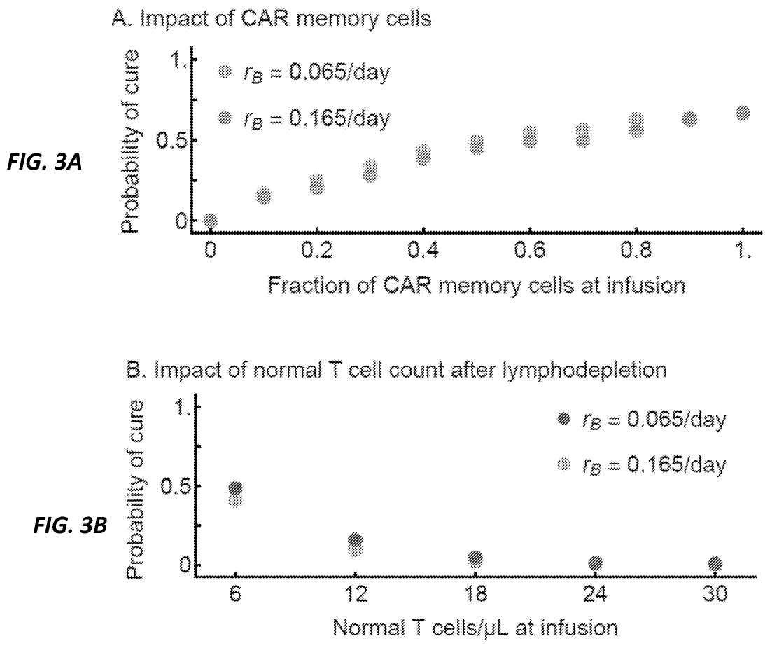

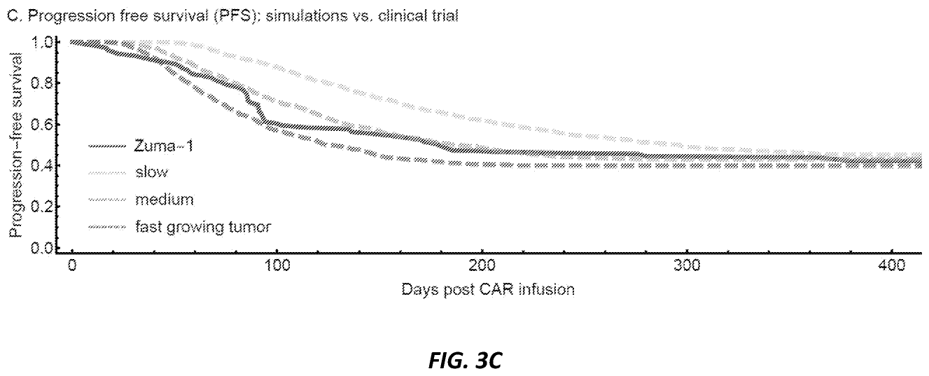

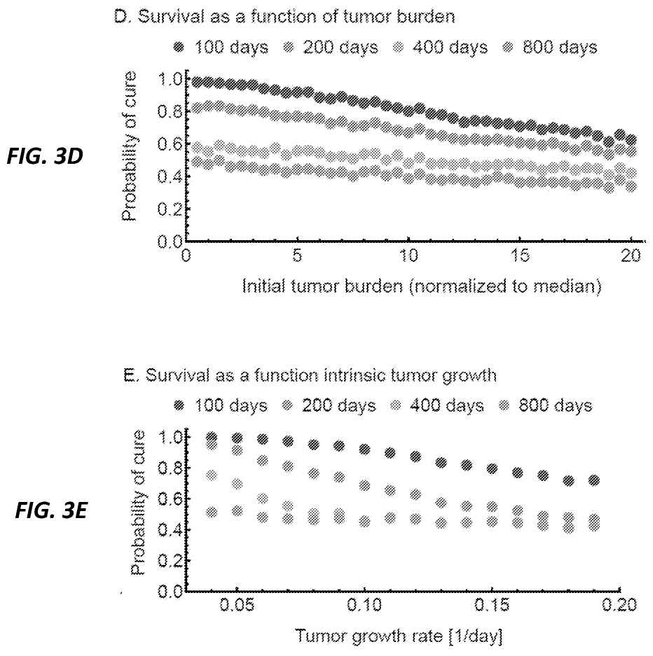

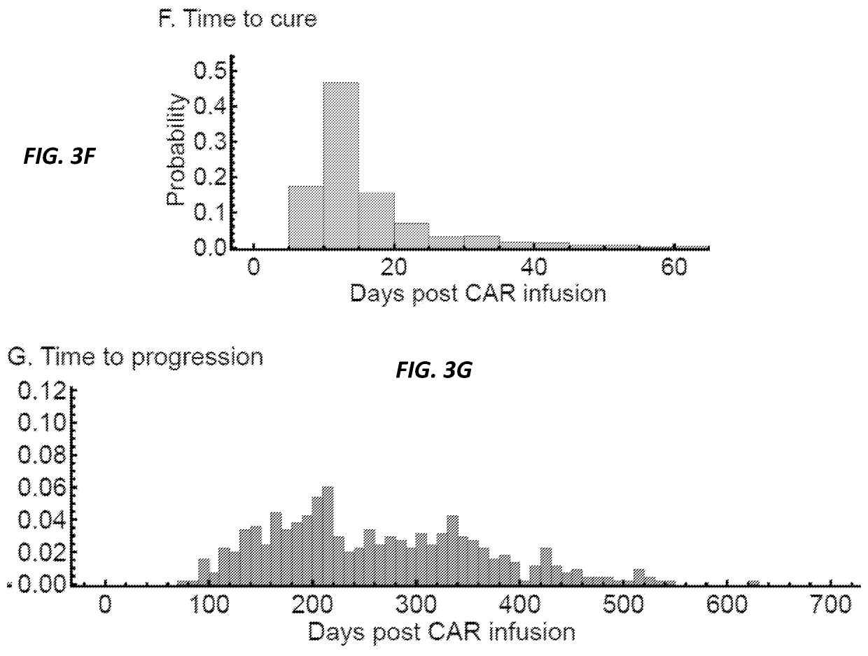

The probability of tumor extinction can be calculated as a function of specific model parameters for a fixed point in time, or integrated for all times. Treatment success (probability of tumor extinction) critically depends on a sufficient fraction of long-term memory/naïve (CCR7+) cells in the CAR product (FIG. 3 A), and on the effectiveness of lymphodepletion that reduce absolute lymphocyte counts (FIG. 3 B). Interestingly, tumor growth rate did not seem to strongly impact the simulation outcomes under the two scenarios of varying initial CAR or normal memory T cells. In particular, we found evidence that, under the circumstances of the median data used for model parameterization, even a pure memory CAR T cell product that can quickly mount an efficient anti-tumor response by rapidly differentiating has a maximal tumor extinction probability near 0.7 (FIG. 3 A). This saturation behavior strongly indicates that other, complementary treatment options should be considered to further increase efficacy.

For a fixed set of mean parameter values (Table 1), tumor eradication or progression can be tracked over time, as shown in FIG. 3 C, assuming various fixed intrinsic tumor growth rates. Remarkably, although we had not used progression-free survival directly as a goal function to find suitable model parameters (Methods, Example 2), our stochastic model can recapitulate the progression-free survival (PFS) of the ZUMA-1 trial.

Differences in timing of events become clear by looking at more fine-grained comparison of survival at distinct time points, as a function of initial tumor burden (FIG. 3 D) and intrinsic tumor growth rate (FIG. 3 E). This analysis shows that higher tumor burden leads to more gradual and less pronounced decline in probability of tumor eradication. In contrast, increased tumor growth rate quickly leads to diminishing chances of tumor eradication.

Hybrid Stochastic Simulations can be Used to Estimate the Influence of Important Parameters on PFS

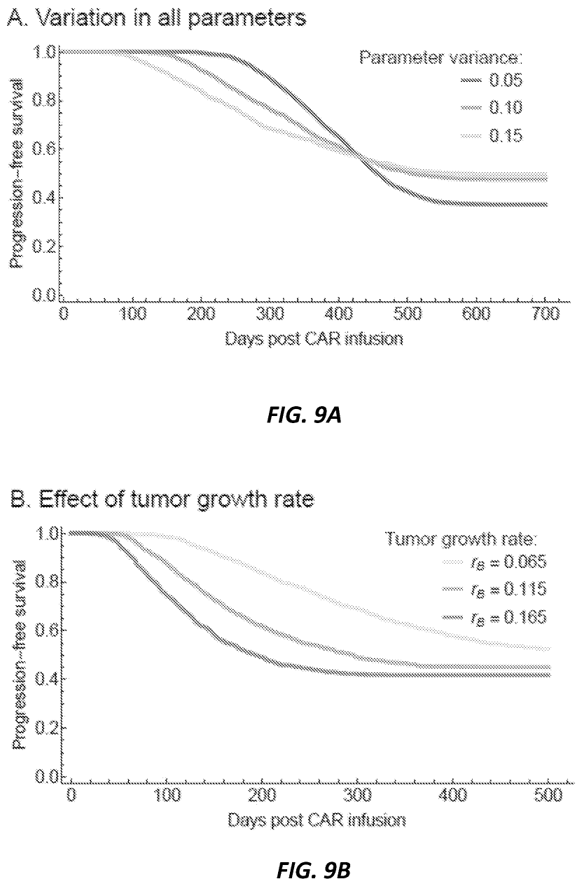

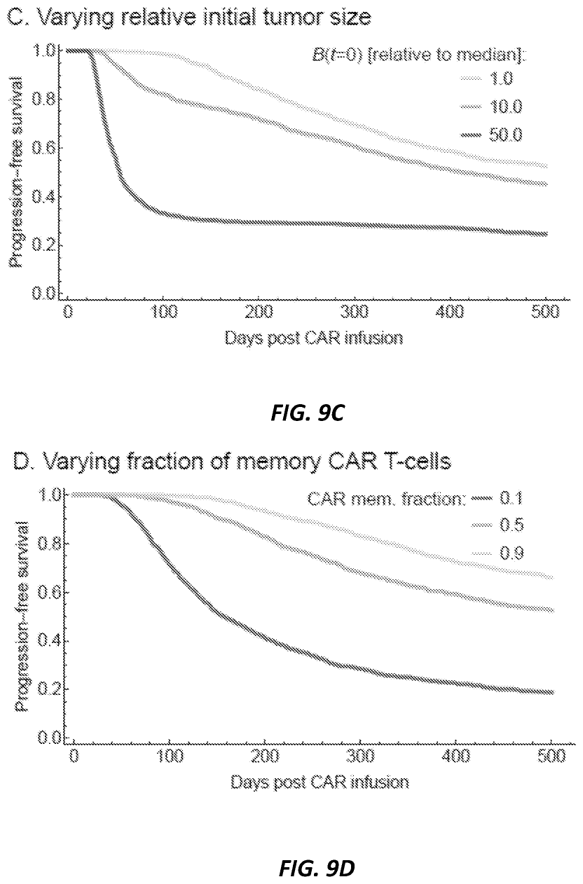

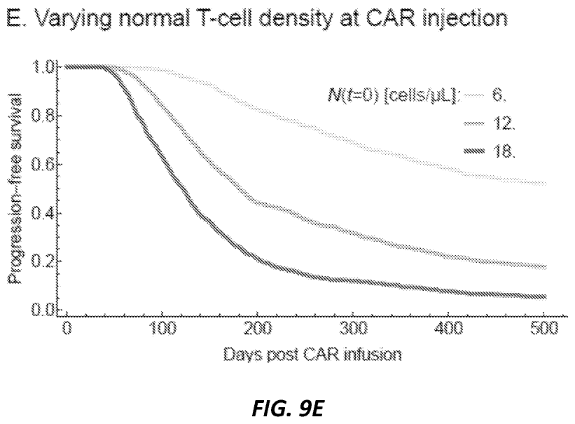

The stochastic representation of the ecological CAR T cell dynamics allowed us to calculate survival curves from a ‘virtual cohort’, with each patient defined by a unique combination of randomly chosen parameter values. We focused on the two scenarios of transient and long-term response. The ‘typical’ case in our simulations, defined by using all median parameter values (Table 1), does not progress until day 180 post CAR infusion. We were thus interested in the overall impact of parameter variation on survival outcomes (FIG. 9 A), assuming that all parameters are normally distributed around the mean value for the median patient, with a variance hyper-parameter calculated as a small fraction of the mean. Increasing parameter variance led to more heterogeneous PFS curves. All differences between simulated PFS curves were statistically significant using a log-rank test due to the large simulated cohort size. Hence, we focused on the magnitude of change in PFS, e.g. at a specific point in time. Our PFS curves show an initial plateau, which stems from the fact that there typically is some initial response to treatment. In the longer term, the PFS curves reflect the inherent stochasticity of tumor extinction or escape.

Larger and Faster Growing Tumors have Worse PFS, with Very Large Tumors Leading to Increased Likelihood of Rapid Progression

Intrinsic tumor properties are the tumor growth rate, r_{B}, and initial tumor burden (B₀, where we report values relative to the median tumor size of 200 cm³). Tumor growth rate increases had a strong detrimental effect on PFS at early timepoints (FIG. 9 B). At day 100, PFS of slow-growing tumors (r_{B}=0.075/day) could be above 90%, whereas PFS of fast-growing tumors (r_{B}=0.175/day) were around 70%. In comparison to the median tumor size, ten-fold larger tumors (B₀=10) showed a drop 70% in PFS at day 100 (from 90% PFS), and to 55% for fifty-fold larger tumors (B₀=50, FIG. 9 C). Note that a fifty-fold larger tumor burden does not need to occur entirely at a single site, but can be spread across multiple lesions.

PFS Declines with Low Levels of CAR Memory Content and Insufficient Lymphodepletion

Next, we asked how PFS is influenced by properties of the CAR and normal T cell population prior to injection. To this end, we considered changes in the fraction of CAR memory fraction at day 0, and variation in the efficacy of lymphodepleting chemotherapy. In concordance with FIG. 2 E, larger differences in PFS were detected when the memory CAR T cell fraction was low (FIG. 9 D). Comparably, the potential failure to effectively lymphodeplete prior to CAR treatment could have drastic effects. Doubling the initial normal T cell population size could halve PFS at day 100 and thereafter (FIG. 9 E).

Cure Events According to Direct CAR T Cell Predation Occur Early, while Progression Events can Occur Late

Our stochastic modeling revealed that cure occurs early; most simulations resulted in cure between days 10 and 35, and we rarely found late cure events up to day 100 (FIG. 3 F). Meanwhile, progression times were distributed over a broad range of time points. Typical progression times occur anywhere between days 20 and 500 (FIG. 3 G). These large differences in time scales occur because cure, as a stochastic tumor extinction event, is much more likely to occur before the effector CAR T cell begins to decline after day 14).

A Second Dose could be Warranted Before Anti-CAR Selection Takes Effect

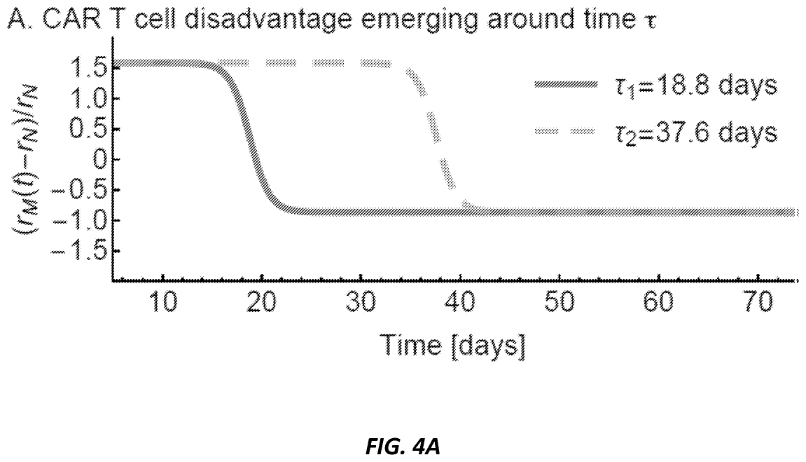

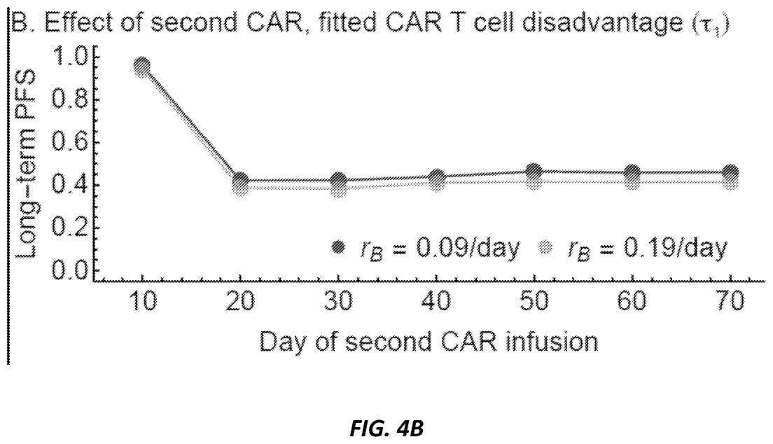

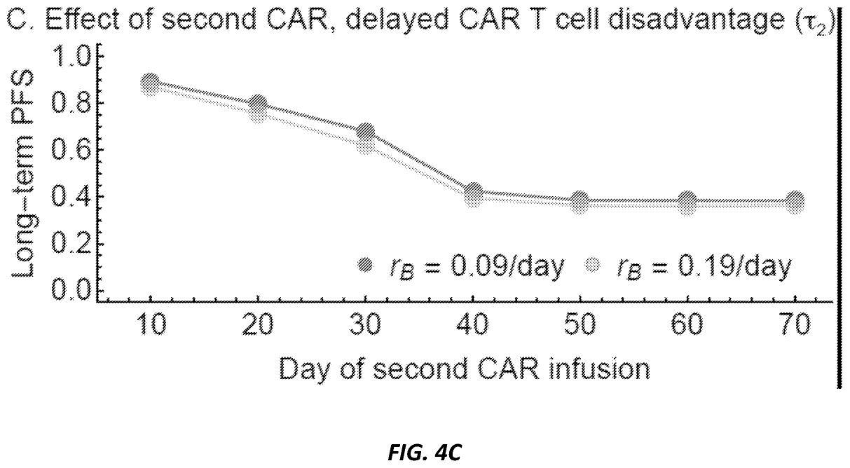

Patients with persistent minimal residual disease, or relapsed patients could receive a second infusion of CAR T cells. Although this second dose could be equivalent or higher to the first dose, there might be little to no effect¹. Our model can attribute this lack of second dose improvement to the selective disadvantage of CAR T cells that is already established in the first weeks after the first dose. Recall that we discovered a switch in CAR memory growth rate, see FIG. 4 A (solid line): our data analysis suggested that the switch occurs around day 19 (in the median patient). Subsequent retreatment after this switch has occurred would then lead to minimal improvement.

The biological mechanism for a CAR growth rate switch might be mounting immunogenicity, as CAR T cells provoke an immune response²¹, which only manifests weeks after lymphodepletion. We hypothesize that this switch is permanent, but could be ultimately resolved using a non-immunogenic CAR.

Second treatment given before an immunogenic effect could lead to improvement, provided that toxicity is low (which might be unlikely due to the rapid sequence of conditioning chemotherapy). Hence, a second dose within 2 weeks following the first might not be feasible (FIG. 4 B). A second dose could become more plausible with a longer time to loss of memory CAR fitness. Thus, we explored a hypothetical scenario of a significantly later switch in CAR growth rate reduction (dashed line in FIG. 4 A). In case of immunogenicity not arising before day 37, a second dose of CAR (together with the required lymphodepleting conditioning) could be given 3-4 weeks after initial infusion, resulting in an observable benefit that is robust to the tumor's intrinsic growth rate (FIG. 4 C).

Discussion

We identified five processes as potential drivers of patient outcomes: the effects of lymphodepleting chemotherapy, the composition of the CAR T cell product (e.g. CCR7+ density), CAR T cell expansion (peak), CAR durability, and tumor burden's impact on expansion. To better understand these processes, we developed a cell population-ecological framework that describes co-evolution between normal T, CAR T (memory and effector), and tumor cells. We established that cure, as a result of tumor extinction, requires a stochastic individual-based description. This stochastic approach then led to predictions and sensitivity analysis of probability of cure and progression-free survival (PFS).

Model calibration and predictions were made using median patient trajectories. We give proof-of-principle that the integration of longitudinal lymphocyte counts with CAR T cell counts and changes in tumor burden can be combined with a mathematical model of CAR T cell population dynamics. Our approach highlights the utility of mechanistic approaches to in vivo CAR T cell therapy dynamics.

Our model confirms the hypothesis that sufficient lymphodepletion is an important factor in determining durable response. We predict that increases in memory CAR T cell fractions (e.g. determined by the fraction of CCR7+ cells) result in expected survival gains. However, diminishing returns in optimizing the infusion bag points to other necessary dynamical quantities that affect CAR expansion and tumor killing, likely impacted by upregulation of inflammatory cytokines, such as IL-7 or IL-15²³. Thus, future modeling that follows from our hybrid stochastic approach should integrate other available signals such as the dynamics and upregulation of homeostatic and inflammatory cytokines.

We made several mechanistic assumptions to approach the broader biological context of CAR T cell therapy. First, we assumed that immune reconstitution varies minimally across patients, but our results demonstrate that this approach contains sufficient feed-back to explain CAR spike and decay. A different approach would be to re-parameterize normal T cell dynamics over time, which could reveal whether variability in normal T cell growth is necessary to explain outcomes.

Second, our model assumes that tumor cell proliferation is independent of tumor burden. However, the tumor growth rate might depend on tumor burden, compared to an innate carrying capacity. In this context, one could explore other sources of tumor burden variability that originate from a logistic dependence of tumor cell proliferation on tumor volume, called proliferation-saturation^{24,25}. Future studies of the stochastic process should carefully consider non-linear relationships between tumor size and growth as a potential dynamic biomarker for the success of CAR T cell therapy.

Third, our probabilistic measure of PFS did not include the evolution of resistance to CAR T cell therapy by genetic or epigenetic mechanisms²⁶. Our results can be seen as conditioned on the non-resistance population, which would add an additional probabilistic modeling layer.

Fourth, normal CD19+ B cells are at negligible levels in the weeks following lymphodepletion. These levels are low until 4-6 months post infusion, implying that their presence is minimal, although they are potential source of target antigen for the CAR. Further, detection of normal CD19+ B cells in circulation long after CAR T is likely evidence that functional CAR T cells no longer persist in the host. It is unclear whether B cells themselves are the driving event for continued persistence. Thus, we assume that non-tumor sources of CD19 do not play a role during the activity of CAR T cells.

We discovered that the CAR expansion rate transitions from a fast to a slow value around a characteristic time T, which we did not anticipate designing the model. This switch affects the entire CAR population but is sufficiently modeled in the slower memory (more stem like) cells, as these less differentiated cells govern the overall dynamics^{27,28}, and could be the result of a mounting immune response against the gene modified cytotoxic cells²⁹. Cytotoxicity could result from rejection of the CAR expressing cells by anti-transgene response³⁰, or anti-CAR T cell immune response against the murine single-chain variable fragment (scFv)¹⁸ that is incorporated in most CD19-targeting CARs. We have obtained estimates of τ that range from 16 to 35 days. This serves as a testable novel prediction that can be studied in animal models, or possibly recapitulated using clinical data together with evidence of mitigated immunogenicity.

As a fundamental characteristic, cure occurred early, progression occurred late. This difference in timing of events indicates that rare patient events in which cure is observed way past 100 days are driven by other mechanisms, such as a vaccine effect in which normal T cells develop increased anti-CD19 activity. Novel mathematical dynamical models of such effects are needed, for which our model can be informative.

CAR T cell therapy can cause severe cytokine release syndrome (CRS), or a characteristic immune cell-associated neurologic syndrome (ICANS). Both are associated with significant co-morbidity and mortality³¹⁻³³, and are characterized by high levels of inflammatory cytokines and, to a lesser extent, by high numbers of CAR T cells in the blood⁷. Both forms of toxicity occur in predictable distributions across patient populations³⁴. There exists an association between CAR T cell expansion and grade 3-4 (severe) ICANS associated toxicities⁷. Elevation of key inflammatory cytokines (IFN-γ, IL-6, IL-1) are also associated with both severe ICANS and CRS. However, a homeostatic cytokine (IL-15) seen at higher levels in responding patients was also associated with toxicity. Several biomarkers were associated with severe ICANS-related toxicity, but not CRS (ferritin, IL2-Rα and GM-CSF)^{35,36}. The occurrence of toxicities could be explained by modeling dynamic cytokine/biomarker levels in periphery together with CAR T cell population dynamics, which could lead to improved understanding of toxicity dynamics. Computational and mathematical tools similar to the ones we developed here could be used to calibrate such a model, assess parameter sensitivities, and make testable predictions of patient outcomes. Thus, our framework presents an important first step towards integration of these mechanisms into a novel quantitative understanding of CAR T cell therapy.

### Example 2

Introduction

In this supplementary text, we give a detailed overview of the data integration and modeling prediction analyses, before we lay out the theoretical foundations of or modeling approach, describe further details of the data analysis and model fitting procedures, and introduce and analyze a hybrid stochastic-deterministic nonlinear modeling approach used for efficient simulations and as a predictive tool. First, we give an overview over the Methods.

Second, we discuss a simple two dimensional stochastic extinction process that could be used to approximate tumor cell extinction as a result of a decaying CAR T cell population. However, this approach does not have the ability to describe the dynamics peak in CAR T cell density.

Third, we motivate and introduce a more comprehensive, four dimensional co-evolutionary frame-work in its mean-field limit. This framework can describe more complex features of both T and tumor cell populations during treatment, in particular CAR peak dynamics.

Fourth, we describe how we analyzed and integrated the available data using the four dimensional framework. The data used consists of (a) median T cell/lymphocyte counts in patients over time, (b) quartile CAR T cell densities in patient over time, and (c) estimates of median tumor burden (derived as tumor volume) over time, which were estimated based on the tumor burden of patients that had progressed at days 30, 60, 90 post CAR administration (beginning of treatment). The data analysis assumes fixed median initial cell densities, and we apply a nonlinear global optimization framework to minimize a pre-defined loss function. This minimization approach leads to possible model parameterizations and provides proof of principle that a four dimensional co-evolutionary framework can be used to describe CAR T cell therapy dynamics.

Last, we introduce a stochastic framework that corresponds to the mean-field model, and which can be used to describe stochastic tumor extinction events. These events are important, as the mean-field approach typically only shows transient tumor reduction-long-term tumor extinction is not stable in the mean-field. To effectively model the fully parameterized four dimensional dynamical system, we introduce and discuss a hybrid stochastic-deterministic framework.

Methods Overview

Clinical Data Integration

We used population-level data from LBCL patients treated on the multi-center ZUMA-1 trial7 and other patients treated at Moffitt, summarized in FIG. 1 C. The qualitative, clinically important dynamics, ranging from no response, to transient response followed by relapse, to long-term response, are shown in FIG. 1 D-F. Based on these qualitative model properties, we sought to evaluate the model's explanatory capacity by integrating it with recent data from clinical studies.

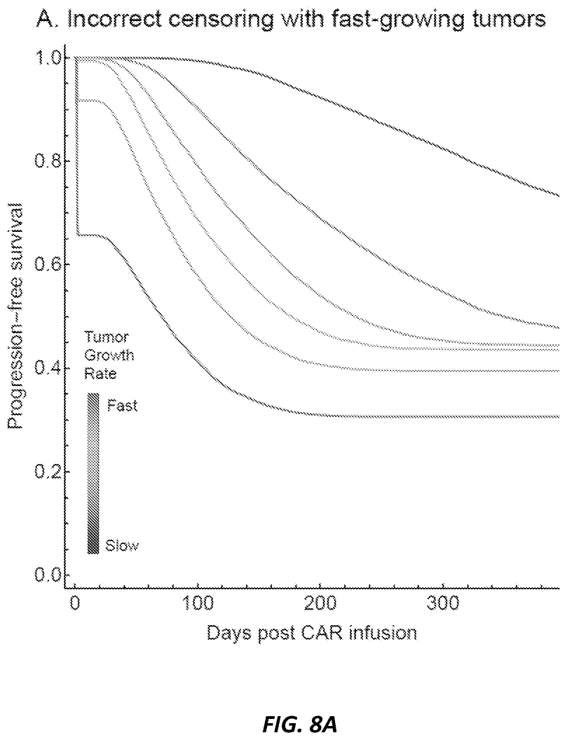

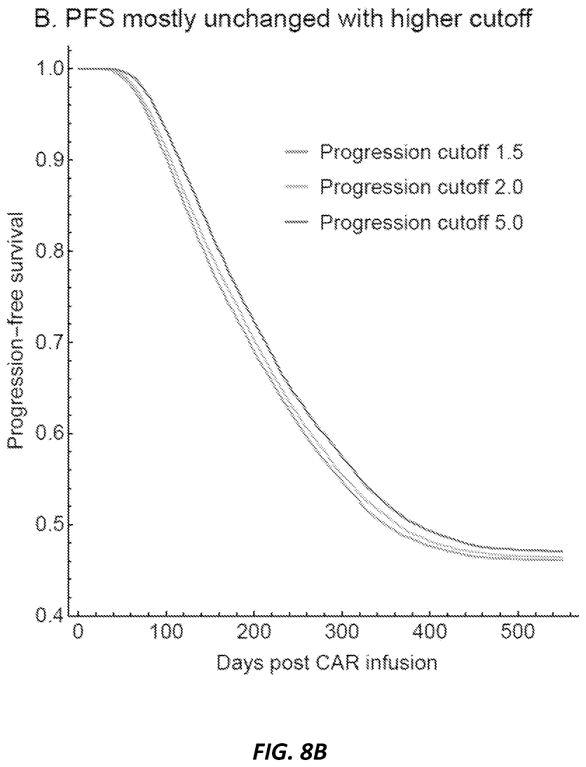

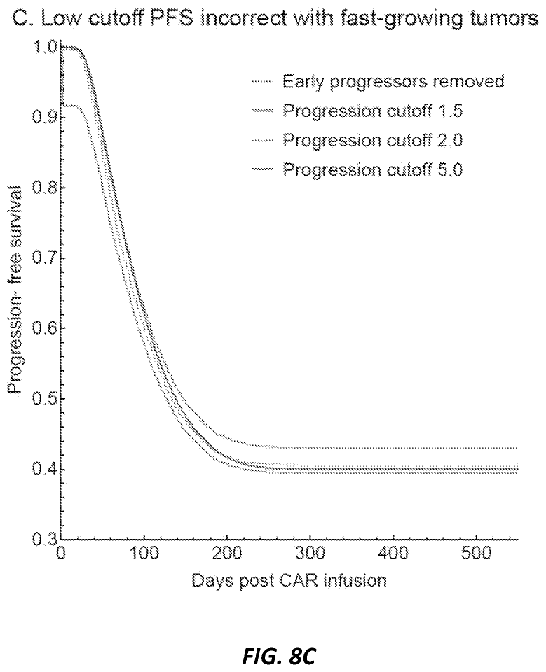

The ZUMA-1 trial reported quartile CAR T cell levels in peripheral blood over five consecutive time points (days 7, 14, 28, 90, 180), which were used to parameterize the mean-field model at the population level (see [5]). We used normal complete blood count-derived absolute lymphocyte counts (ALC, approximating the total T cell compartment), measured in patients receiving CAR T cell therapy (days 0, 5, 7, 14, 28, 90, 180). These counts include CAR T cell density, yet differences in parameters estimates were minimal and no changes in homeostatic ALC were detected, see FIG. 2 A. All peripheral measurements of cells/μL were converted to total T cell counts assuming, 5 L of blood and multiplying by 108, under the assumption that 1% of cells are in periphery at all times and converting μL to L. The normal T cell carrying capacity, K_{N}=500 cells/μL in patients, was estimated from data by Turtle et all. Median tumor volume at day 0 was determined by assuming a spherical lesion, which corresponds to a volume of B0=200 cm3. This tumor size can be converted to 2.0*1011 cells, assuming that 1 cm3 contains 109 cells35. Complete response (CR) was counted as tumor size B(t)=0. Stable disease (SD) was converted to the tumor returning to initial size, B(t)=B0. Progressive disease (PD) was counted as twice the initial tumor size, B(t)=2 B₀. This definition differs from the clinical definition of 1.5 times the initial tumor size as measured by limited and periodic radiographic measurements9, as we sought to avoid incorrect censoring of trajectories described in below and in the SI (FIGS. 8A-8C). Patients in none of the categories (CR, SD, PD) after 1000 days would be defined as undetermined, which did not occur in simulations.

Parameter Estimation

We obtained median parameter values using a nonlinear least squares regression function with the data-variance as weights. The nonlinear optimization problem used for data fitting was solved using the BFGS algorithm from the optimization package in Julia, wrapped around solving the 4D differential equation system (M1)-(M4) (equation system (11) below). For data that was only available in quartiles: we assumed an approximately normal distribution and estimated the variance from the difference between the quantiles. We generated fits to the available quartiles of CAR T cell densities measured at the five consecutive time points available. We fit parameters in equations (M1)-(M2) first, as these decouple from the other equations. Then we fit effector CAR T cell and tumor cell dynamics, equations (M3)-(M4) (see Section “Data Analysis”). The parameter values obtained are shown in Table 1, and their good agreement with median clinical data is shown in FIG. 2 A for the normal T cell dynamics, and in FIG. 2 B for the CAR T cell dynamics.

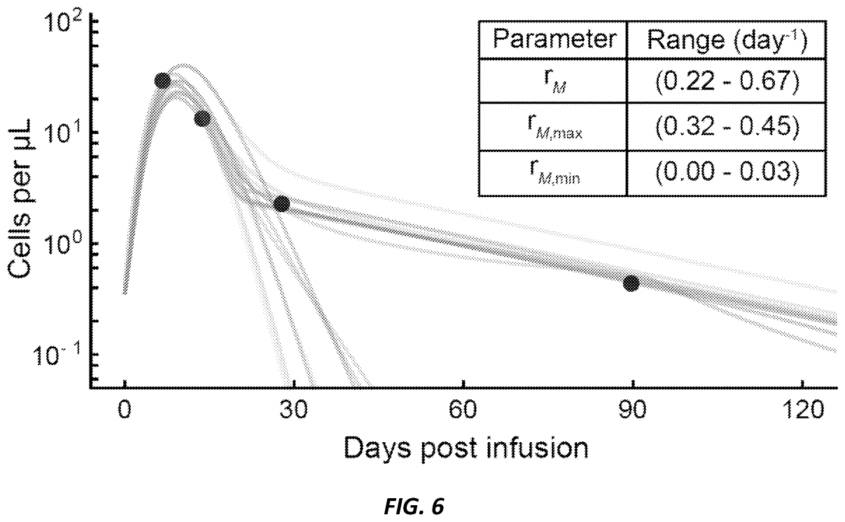

Our model fitting revealed a switch in CAR T expansion rate around a characteristic time, shown in FIG. 2 C and FIG. 6 below. There, we also present possible distributions of the values of the parameters relevant to this switch. Similar distributions were obtained for all parameter values, as discussed below. Some parameter value distributions showed more variability than others. In all time-forward simulations, we used median values if not otherwise indicated.

Model Analysis and Stochastic Time Forward Simulations

We analyzed the deterministic mean field model using linear stability analysis (see Section “Co-evolutionary dynamics among normal, CAR T, and tumor cells”). In the mean-field limit, our parameters lead to eventual cancer progression in every patient. However, many trajectories spend large amounts of time near the tumor-free (B=0) state.

Small populations cannot be adequately captured with a deterministic model, but populations can be driven toward an extinction vortex near small population sizes, stochastic dynamics gain increased importance [1]. This is exemplified in FIG. 2 D, where the exact same set of parameters (Table 1) can result in tumor extinction or progression as two realizations of the same process.

Fully stochastic simulations with a system comprised of billions of cells would take days or weeks to run for a single patient. This is computationally prohibitive since many patient trajectories are needed to gather statistics. To alleviate this issue, while maintaining the accuracy of the stochastic model, we developed a hybrid model that exploits the fact that only certain populations become small and that their fluctuations should only be relevant when a population is below a given threshold. The hybrid model operates in the deterministic limit if the tumor population is above a threshold of S=100 cells and updates stochastically if below the threshold. The other populations are updated deterministically in parallel. Since we are only interested in whether the tumor is eradicated, our model only switches the tumor between deterministic and stochastic model, all other populations remain deterministic. We also investigated the sensitivity of the results for different values of S, ranging from 10 to 1000. We found that our results are robust to the threshold, making the hybrid model a valid approach for solving the complex birth and death model without compromising speed (see Section “Small fluctuations are relevant in small tumor limit”).

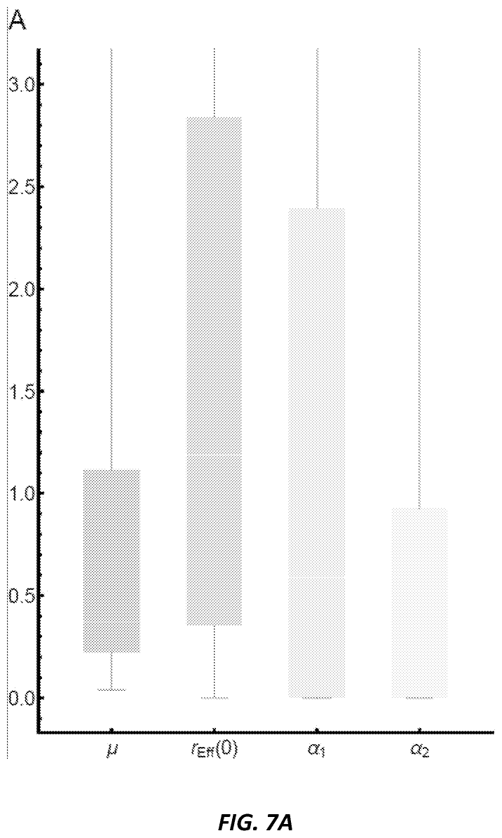

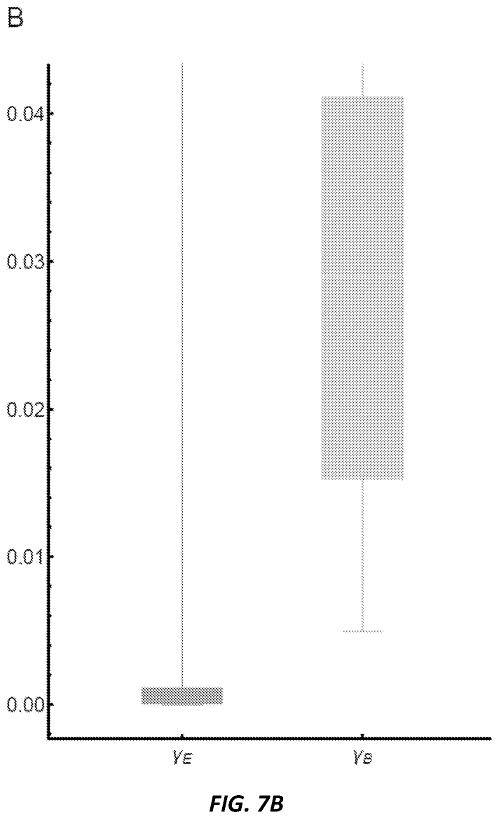

We ran the deterministic system using the built-in solver Rosenbrock23 of the DifferentialEquation package in Julia, and simulated the master equation using a Gillespie algorithm [2]. For a typical simulation result with stochastic outcome, we simulated 1000-10000 virtual patients, each drawn with their own, slightly different mixture of parameters. Parameter variations were achieved using a normal distribution with mean chosen as the median parameter value estimated from model fitting (FIGS. 7A-7B), and variance taken as a fraction of that mean (typically between 5-15%). A patient was counted as cured when their tumor reached 0 cells, which is an absorbing state of the stochastic birth and death process. A patient was counted as progressed when their tumor mass hit the threshold of 2 times their mass prior to treatment. This higher threshold (compared to 1.5 times initial tumor size) was used to avoid the occasional patient's tumor that grows rapidly enough to reach a smaller threshold before decreasing due to treatment, thus causing the simulation to incorrectly classify that patient as progressing. Observed differences in progression times due to alternative tumor size used for progression, e.g. 1.2, 2.0, or 5.0 times the initial tumor burden, are relatively small as can be seen from the following mathematical argument. As CAR cells are depleted and the tumor exceeds B_{threshold}=100 cells, the latter will follow approximately purely exponential growth again. In our simulations, the initial tumor size was B₀=2.00×10¹¹ cells. Thus, the time it takes to reach a threshold relative to initial tumor size is given by T (ß)=(In(ßB₀)/B_{threshold})/r_{B}. Comparing the difference in times with different cutoffs (values of ß), we see that T(ß)≈0.981+0.046 In(ß) T (1.5), where T (1.5) is the clinical measure of progression. We can see by plugging in a few choices for ? that the impact of changing the threshold is minimal (e.g. ß=1.2, 2.0, 5.0 leads to a −1.02%, 1.32%, 5.52% deviation in the progression time compared to 1.5). For example, if progression with clinical definition occurred at day 90, then our cutoff with ß=2.0 would estimate the progression time to have actually occurred at day 91. We used a cutoff of 2.0 to avoid potential incorrect censoring of trajectories with faster-than-average growing tumors, which only resulted in a minor shift in time to progression (see FIGS. 8A-8C).

Stochastic Tumor Cell Extinction with a Simple CAR-Decay Model

First, we discuss an approach that does not consider CAR T cell expansion or feedback by tumor antigen, but describes the essence of CAR T cell predation and provides an intuition for tumor cell extinction driven by CAR T cells. Let us consider two cell populations; CAR T effector cells E(t) that kill tumor, and malignant B cell B(t). In the simplest setting, we assume that tumor killing cells E have their maximal value at time t=0, and that these cells obey a simple death process. That is, they will go extinct with probability 1 eventually:

$$E {\to }^{{d}_{E}} ∅ . \qquad ( 1 )$$

Meanwhile, these Tumor Killing Cells Contribute to Tumor Cell Death, but the Tumor Follows a Birth and Death Process

$$B {\to }^{{b}_{B}} B + B , \qquad ( 2 )$$

$$B {\to }^{{d}_{B}} ∅ , \qquad ( 3 )$$

however, we would have to assume that the tumor cell birth rate is a function of E(t). The simplest approach, however, would be to assume a time scale separation, and set d_{B}=δ_{B} E₀. Then, we can write down the following master equation for the Markov process that governs tumor cell count over time (using f=df/dt notation)

\dot{P}_{B}(t)=b_{B}(B−1)P_{B-1}(t)+d_{B}(B+1)P_{B+1}(t)−(b_{B}+d_{B})BP_{B}(t) (4)

where P_{B}(t) is the probability to find the system in state B at time t. Using the generating function approach [3], we can obtain the following partial differential equation (PDE) for the generating function F (t, x):=B Pₘ(t) x^{B} (whereby ∂ₓ means partial derivative with respect to x)

F(t,x):=Σ_{B}Pₘ(t)x^{B} (whereby ∂ₓ means partial derivative with respect to x)

F=b_{B}(x²−x)∂ₓF+d_{B}(1−x)∂ₓF (5)

subject to boundary conditions F (t, 0)=P_{B=0}, and F (t, 1)=1, and the initial condition F (0, x)=x^{B}0. Here, B₀ is the initial tumor size, and P_{B=0} is the tumor extinction probability, which turns out the be the quantity we are interested in. The PDE for F is of Lagrange type and can be solved exactly

$$F ( t , x ) = {( \frac{( x - 1 ) {d}_{B} {e}^{( {b}_{B} - {d}_{B} ) t} - {b}_{B} x + {d}_{B}}{( x - 1 ) {b}_{B} {e}^{( {b}_{B} - {d}_{B} ) t} - {b}_{B} x + {d}_{B}} )}^{{B}_{0}} , \qquad ( 6 )$$

which then yields the probability of tumor extinction at time t:

$${P}_{B = 0} ( t ) = F ( t , 0 ) = {( \frac{{d}_{B} - {d}_{B} {e}^{( {b}_{B} - {d}_{B} ) t}}{{d}_{B} - {b}_{B} {e}^{( {b}_{B} - {d}_{B} ) t}} )}^{{B}_{0}} . \qquad ( 7 )$$

Since we made the assumption of a constant tumor death rate, we can now heuristically calculate the probability of cure as the conditional probability of two independent events, i.e. the product of P_{B=0} and the probability that the CAR T cell population E does not go extinct. For the simple death process assumed for E, we obtain the extinction probability

P_{E=0}(t)=(1−e^{-d}^{_{E}}^{t})^{E}^{₀}, ((8)

and thus could approximate the likelihood of tumor extinction, conditioned on CAR T cell survival, as P_{B=0}×(1−P_{E=0}).

However, in a more realistic setting, the tumor death rate is time dependent via its dependence on

the number of tumor killing T cells, the CAR T cell birth rate is not zero, and its birth and death rates are also time dependent. This implies that one can find a higher-dimensional process that removes this explicit time-dependence [4]. In the following, we identify a four-dimensional system that models T and tumor cell co-evolution more comprehensively.

Co-Evolutionary Dynamics Among Normal, CAR T, and Tumor Cells

A comprehensive mathematical model of tumor cell and CAR T cell evolution has to include non-monotonic changes of the total CAR T cell population, as well as the ability to adjust the relative contribution of CAR T cells that replicate, and CAR T cells that kill tumor at any point in time. This could be achieved by assuming that changes in the CAR T cell population are subject to a time-varying function. In the following, we develop a model that attributes such time dependence to a third T cell population, namely normal T memory cells.

To motivate a comprehensive approach modeling CAR T cell and tumor cell dynamics, we define the desired properties the model should contain. First, tumor growth should be countered by CAR T cell predation, such that if predation occurs at a higher rate, the tumor shrinks. Second, tumor, or B cell antigen (CD19) should affect CAR T cell expansion. Third, the CAR T cell product's volume (population size) and composition should be integrated, leading to a CAR T cell population that is sub-divided into at least two populations, for example (central) memory and effector CAR T cells. Fourth, the overall CAR T cell population should have the ability to expand and contract, both as a result of temporally changing signals within the patient.

Motivation

We present here a game-theoretic, or co-evolutionary approach that leads to a mechanistic mathematical model, which can be used to describe the likelihood of successful treatment. As a key property, treatment success should be proportional to the CAR T cell population's ability to expand before its inevitable contraction. We contrast our approach to a simple statistical analysis that does not capture CAR expansion, see FIG. 5 A, which shows the inner quartiles of the trial ZUMA-1 trial [5]. The characteristic maximum in observed CAR T cell concentration occurs about a week post-infusion. In addition, on can observe that three months post-infusion, the CAR population is still present in the peripheral blood in more than 50% of patients. This can be approached as a simple decay problem, and one can ask whether it is likely that exponential decay governs the dynamics, or whether other time scales matter. In FIG. 5 B we compare the results (P-values) of linear regression analysis to the log-linear or log-log transformed median CAR T cell concentrations. In this context, it becomes apparent that exponential decay does not fully capture the dynamics. Rather, CAR T cells seem to exhibit a different half-life and long-term behavior; the long-term dynamics seem to be best approximated by a time dependence of the form ˜t^{-ß} (see FIGS. 5 C, D, E). This might indeed be indicative of some form of memory from the initial time point (or before), thus it highlights the role of the system's state before CAR administration. To motivate our assumptions, we first state the following observations:

If lymphodepletion of normal T cells does not occur, the CAR T cells do not expand in vivo. Implication: A carrying capacity, or other feedback of the overall lymphocyte count must exist, which is related to the total T cell count over time. Otherwise, CAR T cells would always expand.

The CAR T cells have an initial spike in concentration before decreasing and leveling out to a constant (which may or may not be zero). Implication: The amount of space, or niche available to CAR T cells changes over time.

Influences of dynamic cytokine changes or other factors impact both normal and CAR T cells, and introduce a form of memory. Implication: Some cellular reaction rates, such as the CAR T cells' growth rate, could be time dependent explicitly. Note, however, that complete clinical response is possible with undetectable CAR T cell levels in the long term.

We here propose a mechanism that drives the early of CAR T spike, followed by decay. In this work we postulate that the mechanism that drives the dynamics of CAR cells is competition, or co-evolution, with normal T cells, as well as the dynamics feedback of the targeted tumor cell population.

Modeling Re-Emergence of Normal T Cells, CAR Dynamics, and CAR-Tumor Interactions

Our approach is motivated by two key observations. First, chemo-depletion of T cells seems to be required for proliferation of CAR T cells. This observation implies that there is an innate carrying capacity shared by both types of T cells (wild-type and CAR). The initial depletion is necessary to give CAR cells an ability to proliferate. The nonlinear nature of this dynamic behavior indicated that there are compartments of CAR T cells that play distinct roles, such as naïve, central memory and effector cells.

Second, after a peak at around 7 days post injection, CAR levels begin to decay. This observation could imply that a wild-type/normal T cell population has an overall advantage—otherwise the long-term levels of CAR would not tend to zero. These observations lead to the following model assumptions.

Assumptions

We conceptualized a model of CAR T cell therapy based on four key assumptions. First, we consider two sub-populations of CAR T cells. We combine the compartments of naïve and central memory cells into one, which we call “memory” CAR T cells, M. When presented with antigen, M cells differentiate into a second compartment of effector and effector memory CAR T cells, which we call “effector” CAR T cells, E. These cells do not divide, and target and kill antigen presenting tumor cells, B.

Second, we introduce co-evolution, or competition, in the naïve/memory T cell compartment. To this end we consider the normal T cell population, N. The populations M and N can inhibit each others' expansions, but at different rates. That is, we assume negative feedback mechanisms between M and N, their net growth rates depend on the total amount M+N, for which one can introduce carrying capacities (K_{M}<K_{N}). In this sense, CAR T cells are maladapted: normal T cells have the propensity to expand to higher numbers during immune reconstitution. Hence, memory CAR T cells eventually will be selected against (unless other long-term memory mechanisms would be considered) and decay, but may get a head start and expand, due to lymphodepletion. This expansion is modeled by asymmetric differentiation of M cells into E cells.

Third, we assume that asymmetric differentiation from memory to effector CAR T cells is antigen-dependent. CAR effector cells kill tumor cells at a high rate, and at the same time these effector cells have a finite life-span. We introduce a predator-prey interaction term between E and B that defines the killing rate of the tumor cells when engaged by a CAR effector cell. For simplicity, we assume a linear (Holling type I) predation rate. Other, more complex interaction terms could be considered at the cost of additional parameters.

Last, we assume simple exponential growth of the tumor cell population B∝e^{r}B t. These assumptions and relationships can be cast as the following system ordinary differential equations (ODEs):

\dot{N}=N(r_{N}−a₁₁N−a₁₂M), (9a)

\dot{M}=M(r_{M}−a₂₁N−a₂₂M), (9b)

Ė=r_{E}(B)M−γ_{E}BE−d_{E}E, (9c)

\dot{B}=r_{B}B−γ_{B}BE. (9d)

With initial values of N₀, M₀, E₀, B₀, respectively. Here r_{M} and r_{N} are the net growth rates of CAR and normal memory T cells, respectively. The coefficients αᵢⱼ are the interaction rates that determine the magnitude of negative feedback that the j subtype has on the i subtype. r_{E} is the asymmetric differentiation rate and d_{E} is the CAR effector death rate. The rates γ_{E,B} are the effector-tumor interaction rates, where an effector or tumor cell dies upon interaction, respectively. Finally, r_{B} is the tumor's intrinsic net growth rate.

Carrying Capacity Formulation

A general formulation of interaction involves a competitive Lotka-Volterra model with interaction coefficients αᵢⱼ. However, it is very difficult to infer these parameters from biological data. A simplifying assumption involves scaling these coefficients αᵢⱼ=1/Kᵢ, i.e. introducing a carrying capacity that each subtype would have in the absence of the other subtype. Hence, by redefining the parameters αᵢⱼ=rᵢ/Kᵢ, we arrive at

$$\dot{N} = {r}_{N} N [ 1 - {( \frac{N + M}{{K}_{N}} )}^{β} ] , \qquad ( 10 a )$$

$$\begin{matrix}\dot{M} = {r}_{M} M [ 1 - {( \frac{N + M}{{K}_{M}} )}^{β} ] , & ( 10 b )\end{matrix}$$

$$\dot{E} = {r}_{E} ( B ) M - {γ}_{E} BE - {d}_{E} E , \qquad ( 10 c )$$

$$\dot{B} = {r}_{B} B - {γ}_{B} BE . \qquad ( 10 d )$$

Preliminary optimization of the model parameters showed that ß→0, and that rᵢ is large. Rescaling

rᵢ=rᵢ/ß(i=N, M) and letting ß→0 we obtain the following form for our model

$$\dot{N} = - {r}_{N} N ln ( \frac{N + M}{{K}_{N}} ) , \qquad ( 11 a )$$

$$\dot{M} = - {r}_{M} M ln ( \frac{N + M}{{K}_{M}} ) , \qquad ( 11 b )$$

$$\dot{E} = {r}_{E} ( B ) M - {γ}_{E} BE - {d}_{E} E , \qquad ( 11 c )$$

$$\dot{B} = {r}_{B} B - {γ}_{B} BE , \qquad ( 11 d )$$

which is the form of the mean-field model we use for the remainder of the text.

Steady States

Since N and M are only dependent on each other, we can consider them separately. If M*=0, then N*=0, K_{N}. Suppose M*=0, then if N*=0 we have M*=K_{M}. A coexistence state is only observed in the case K_{N}=K_{M}. Since indefinite persistence of CAR T cells was not observed clinically, we conclude that K_{N}>K_{M} is clinically/biologically realistic. This assumption helps to limit the parameter region.

We note that there are no coexistence states. The stability and states can be summarized as follows:

Unphysical solution: (0, 0, 0, e^{r}B t)—the T cell-absent state. This state is always unstable, but unphysical in the sense that there would have to be an absence of normal T cells at any time.

Failed treatment: (K_{N}, 0, 0, e^{r}B^{t})—the CAR cells are depleted and the tumor is not eliminated, leading to exponential tumor growth. This state is stable if K_{N}>K_{M}.

Cured patient:

$$( 0 , {K}_{M} , \frac{{K}_{M} {r}_{B} ( 0 )}{{d}_{E}} , 0 )$$

—the CAR cells eliminate the tumor and outcompete the wild-type to become the resident population. This case is also unrealistic—we expect the normal T cells to have an innate advantage due to a deeper stem and progenitor cell pool of normal cells. This is stable if

$${K}_{M} > max ( {K}_{N} , \frac{{d}_{E} {r}_{B}}{{γ}_{B} {r}_{E} ( 0 )} ) .$$

Stable Patient:

$$( 0 , {K}_{M} , \frac{{r}_{B}}{{γ}_{B}} , {B}_{stable}^{*} ) , \text{} where {B}_{stable}^{*} satisfies {r}_{E} ( {B}_{stable}^{*} ) {γ}_{B} {K}_{M} - {r}_{B} ( {γ}_{E} {B}_{stable}^{*} E + {d}_{E} ) = 0. \qquad ( 12 )$$

Here, the CAR-tumor interactions dynamically stabilize the tumor. This case is also unrealistic{

we expect the wild-type population to be present. Nonetheless, this state is stable if

$${K}_{M} > max ( {K}_{N} , \frac{{γ}_{E} {r}_{B}}{{γ}_{B} {r}_{E}^{′} ( {B}_{stable}^{*} )} ) .$$

Tumor growth: This state is slightly different than the states considered so far in that it is not a “fixed point”. Ultimately, we are concerned with tumor growth. If the maximum value of E is such that B>0, then we have an unsuccessful treatment outcome. Hence, if

$$E ( t ) < {E}_{max} = \frac{{r}_{B}}{{γ}_{B}}$$

the tumor will grow (note that this bounds solutions away from the stable patient outcome.)

We can see that under the assumption K_{N}>K_{M}, the only long-term states are failed treatment and eventual tumor growth. Note that this includes cases in which the tumor shrinks temporarily. In the deterministic setting considered so far, the tumor cell population can become arbitrarily small and spend long times in a regime near 0, where random cell death events could lead to the elimination of tumor. These small-population size effects cannot be adequately captured with a mean-field ODE model. Hence, we will eventually turn to a stochastic model formulation. Before we discuss the stochastic system equivalent to the mean-field dynamical system, we first ask under which conditions CAR T cell densities can exhibit non-monotonic temporal behavior.

Conditions for Non-Monotonic CAR T Cell Population Dynamics

In FIG. 5 A, we see that the CAR levels reach a maximum if we factor in the initial CAR density, followed by decay. We here derive the necessary conditions for this peak to occur, in the general case, and then for the carrying capacity model as a corollary.

Let us first consider the CAR memory compartment. For a spike in CAR to occur, we require there to exist an Npeak, Mpeak such that \dot{M}=\ddot{0} and \ddot{ }\ddot{M}<0. This leads to

$${M}_{peak} = \frac{{r}_{M} - {a}_{21} {N}_{peak}}{{a}_{22}} . \qquad ( 13 )$$

Differentiation of Eq. (9b) leads to

\ddot{M}=\dot{M}(r_{M}−a₂₁N−a₂₂M)−M(a₂₁\dot{N}+a₂₂\dot{M}) (14)

Evaluating this at Npeak, Mpeak leads to

\ddot{M}=a₂₁\dot{N}Mₚₑₐₖ (15)

Hence, a peak will exist \dot{N}>0 at that point. Furthermore, we require Mpeak>0, which implies r_{M}<a₂₁Nₚₑₐₖ. Looking at Eq. (9a) at the peak yields

$$\dot{N} = N ( {r}_{N} - \frac{{N}_{peak} Δ + {a}_{12} {r}_{M}}{{a}_{22}} ) , \qquad ( 16 )$$

where Δ=a₁₁a₂₂−a₂₁a₁₂ is the determinant. This leads to the requirement that

a₂₂r_{N}>NₚₑₐₖΔ+a₁₂r_{M}. (17)

In the carrying capacity case, we note that Δ=0 and so our conditions reduce to K_{N}>K_{M}>Nₚₑₐₖ. We recognize the first inequality as the requirement that the CAR memory only state is unstable.

Data Analysis

We used quartile data of 101 patients from the ZUMA-1 trial, as published by Neelapu et al. (2017) [5]. This data set contains total peripheral CAR T cell concentrations, from peripheral blood measurements, recorded at days 7, 14, 28, 90, and 180 post CAR injections. These 15 data points, in combination with normal T cell/lymphocyte counts obtained from some of these patients independently at Moffitt Cancer Center at day 0, 5, 7, 14, 28, 90, 180, as well as using clinical response data as a proxy for tumor size, were used to fit the parameters of the T cell-CAR T cell and tumor cell dynamics model in its deterministic limit.

To arrive at tumor size estimates, we assumed the following breakdown of clinical responses, respectively at time t=30, 60 and 90 days post CAR injection. Complete response (CR) was counted as tumor size B(t)=0/no detectable tumor. Stable disease (SD) was counted as the initial tumor size, B(t)=B₀. Progressive disease (PD) was counted as twice the initial tumor size, B(t)=2×B₀.

Note also that the breakdown of the total CAR T cell population into compartments M and E was only available at time of CAR administration. Hence, when fitting these data, one must either make assumptions on the phenotype breakdown at each available time point, or compare the model solution (M+E)(t) concentration against the total CAR T data. We separated the data into subsets assuming fixed fraction of CAR memory cells fₘₑₘ=M(M+E), and thus were able to treat the compartments M and E as separate data/modeling functions.

To fit the data, we implemented a BFGS constrained optimization routine using Julia's built in optimization and differential equation solvers to determine the optimal parameters which minimize our predefined loss function [6, 7]. We used a weighted-least squares function, where the weights are the variance of the data at each time point. Using the quartile data only, we assume that the distribution of CAR T cell levels at each time point is normally distributed about the median and calculate an estimate of the variance from the interquartile range:

$${σ}^{2} \approx {( \frac{{Q}_{3} - {Q}_{1}}{1.35} )}^{2} . \qquad ( 18 )$$

We first fit the normal and memory CAR T cells, since the two compartments M and E are decoupled from the other two (E, which depends on M and B, and B, which depends on E). The normal T cell carrying capacity, K_{N}=500 cells/μL in patients, was estimated from [8]. The loss function used is

$${θ}_{N , M} = {λ}_{0} \sum \frac{{[ M ( {t}_{i} ) - {\hat{M}}_{i} ]}^{2}}{{σ}_{M , i}^{2}} + {λ}_{1} \sum {[ N ( {t}_{i} ) - {\hat{N}}_{i} ]}^{2} , \qquad ( 19 )$$

where the λ's are tunable weights that can be used to adjust the loss function landscape. Different A's lead to different optimal parameter sets. We generated trajectories by using the three quartiles and using the assumption that the memory fraction fₘₑₘ was fixed over time. We chose fₘₑₘ=[0.1, 0.5, 0.9], which led to a total of nine trajectories.

Of note, the initial CAR T cell spike near day 7, followed by a fast decay, can be well captured by three fitted parameters r_{N}, r_{M}, K_{M}, in principle. However, the data showed a slowing-down of CAR decay after day 14—some patients still had observable CAR levels 6 months post-infusion. This behavior could not be captured with these three parameters only. To account for this, we introduced two additional parameters that govern a switch in the value of the CAR growth rate r_{M}. As K_{M} is intricately linked to the long-term stability of M and N, it made more sense to put this time-dependence into r_{M}. We introduced the function

$${r}_{M} ( t ) = \frac{{r}_{M , max} - {r}_{M , min}}{1 + {e}^{t - τ}} + {r}_{M , min} . \qquad ( 20 )$$

The improvement can be seen in FIG. 6 where the single parameter for r_{M} (pink trajectories) does not adequately capture the slow decay, post-expansion. In contrast, the introduction of a growth rate switch (blue trajectories) can account for this behavior.

Many functional forms can be given for the antigen/tumor size-dependent production rate of effector CAR T cells, r_{E}(B). Clinical trial data and in vitro experiments show that more antigen presentation leads to an increase in differentiation into effector cells [9, 10, 11]. We modeled this as the following piecewise-linear function

$${r}_{E} ( B ) = {r}_{E} ( 0 ) [ 1 + {α}_{1} min ( \frac{B}{{B}_{0}} , {α}_{2} ) ] . \qquad ( 21 )$$

To obtain the parameters in the E and B compartment, we introduced a second loss function

$${θ}_{E , B} = {λ}_{0} \sum \frac{{[ E ( {t}_{i} ) - {\hat{E}}_{i} ]}^{2}}{{σ}_{E , i}^{2}} + {λ}_{1} {\sum }_{i ∈ \{ 30 , 90 , 180 \}} [ B ( t ) - \hat{B} ( t ) ] + {λ}_{2} f ( \vec{p} ) , \qquad ( 22 )$$

where the fitted parameters for M, N are used to solve for the remaining parameters. Here, f(p) represents additional constraints imposed based on what is expected biologically. In this case, quartile CAR levels indicates that CAR population initially expands, so we introduce a penalty f (p)=max[(d_{E}+γEB₀)E₀/M₀−r_{E}(B₀), 0] to enforce this condition.

In summary, the mean-field ODE model was used for this non-linear constrained optimization approach to find best fits (of which there are potentially many). The parameter values obtained are shown in Table 1. In FIGS. 7A-7B, we show the distribution of parameter values obtained from using different lambdas which can be one of the following values from the set {0.1, 1.0, 10}. We stress here that this will not, nor expect to yield unique parameters. Different A change the shape of the loss function. We used the fits in conjunction with how well they fit the CAR data (the only data for which we have temporal measurements) as a secondary comparison. Therefore, though most parameters used in the table fit somewhere in the distribution, not all of them need fit there to get good agreement with the data. In particular, the a's were the least well-behaved parameters obtained in the fit. This is not entirely unexpected, it is mostly tied to the size of B, for which we have the least amount of information. The term containing information on B contained three typical scenarios at the follow-up days 30, 90, 180. Either complete response (CR,CR,CR), initial response (CR, PD, PD) or progression (PD, PD, PD) where we defined PD=2.0B₀ and CR=0. In section “Progressive disease criterion and its impact on K-S curves”, we show that the impact of shifting progression from the clinical definition of 1.5 to 2.0 times the initial tumor size can be expected to small. However, with smaller cutoffs comes the risk of false progressions (see section “Progressive disease criterion and its impact on K-S curves” and FIGS. 8A-8C).

In the next section, we introduce a stochastic framework that considers individual cellular events. We used the model parametrization presented in Table 1, for which some of the values had to be scaled to represent individual cellular events, as indicated by the additional column.

Small Fluctuations are Relevant in the Small Tumor Limit

We have discussed the stability and fixed points of Eq. (9). This system describes initially successful treatment and a peak in CAR concentration over time. However, as normal lymphocytes reemerge and reaches a homeostatic level, CAR decays, the tumor eventually grows back and relapse occurs deterministically in the mean-field framework. Although this scenario is plausible, it is still one of several outcomes.

Complete durable responses have been observed in the clinic (typically without detectable CAR levels long-term). When the tumor population becomes small, stochastic effects (fluctuations) become relevant—the tumor might be subject to an extinction vortex [13]. These chance extinction events can lead to tumor extinction. Of note, our modeling framework implies that stochastic extinction is a necessary requirement for durable response, if CAR T cells do not persist indefinitely. In what follows, we will propose a stochastic framework that in the large population limit will converge to the deterministic dynamics.

Stochastic Dynamics

Let N, M, E, B now be the cell numbers of wild-type, CAR memory, CAR effector and tumor cell populations, respectively. We define the following birth events,

$$N {\to }^{{r}_{N}} N + N , \qquad ( 23 )$$

$$M {\to }^{{r}_{M}} M + M , \qquad ( 24 )$$

$$M {\to }^{{r}_{E} ( B )} M + E , \qquad ( 25 )$$

$$B {\to }^{{r}_{B}} B + B , \qquad ( 26 )$$

and the following death events

$$N {\to }^{{δ}_{N} ( N , M )} ∅ , \qquad ( 27 )$$

$$M {\to }^{{δ}_{M} ( N , M )} ∅ , \qquad ( 28 )$$

$$E {\to }^{{δ}_{E} ( B , E )} ∅ , \qquad ( 29 )$$

$$B {\to }^{{δ}_{B} ( B , E )} ∅ . \qquad ( 30 )$$

We can now calculate the transition rates to move state i to state j. We choose a time interval small enough such that only a single event occurs (all other events occur on the order O(Δt)²). Defining T (N±1, M, E, BIN, M, E, B)=TN± and noting that rᵢ can depend on other populations we have the population transition rates

$${T}_{i}^{+} = {r}_{i} i , i = N , M , E , B , \qquad ( 31 )$$

$${T}_{N}^{-} = {δ}_{N} N = {r}_{N} N ln ( \frac{N + M}{{K}_{N}} ) , \qquad ( 32 )$$

$${T}_{M}^{-} = {δ}_{M} M = {r}_{M} M ln ( \frac{N + M}{{K}_{M}} ) , \qquad ( 33 )$$

$${T}_{E}^{-} = {δ}_{E} E = {γ}_{E} EB + {d}_{E} E \qquad ( 34 )$$

$${T}_{B}^{-} = {δ}_{B} B = {γ}_{B} EB . \qquad ( 35 )$$

Denoting P (N−1, M, E, B, =P_{N-1}, the corresponding master equation is given by

$$\frac{\partial P}{\partial t} = {T}_{i - 1}^{+} {P}_{i - 1} + {T}_{i + 1}^{-} {P}_{i + 1} - ( {T}_{i}^{+} + {T}_{i}^{-} ) P , \qquad ( 36 )$$

where i=N, M, E, B.

A shortcoming of an exact simulation of this four dimensional stochastic framework is the computing time required for typical tumor size and lymphocyte count, which are in the orders of millions to billions. The number of events to observe a complete response is when the number of tumor cells approaches

0. The minimum number of stochastic individual cellular events needed then is clearly B₀, where B₀ is the initial number of tumor cells (see Table 1).

In practice, the total number of simulation events needed to approach tumor extinction will exceed B₀ by a substantial amount. On conventional CPUs, the amount of time it takes for a single run can vary from hours to days. The issue is that the transition rates are so high, which means that the time to a possible next event are very short. A way to overcome this obstacle was proposed by Gillespie himself, who suggested the method known as “tau-leaping” [14]. This method is similar to a forward Euler method for a continuous system, except that the update is taken as a Poisson distributed random variable. The time τ is given as a fixed step-size, which alleviates the events occurring rapidly, but error is introduced as the simulation is no longer exact (populations are assumed to be constant in the time interval [t, t+τ]. This method works well in many cases, unless if the cell populations exhibit substantially different scales.

Suppose there are two populations, one small (stochastic fluctuations are relevant) and the other very large (fluctuations are not relevant). In this case, tau-leaping could potentially lead to large error if a step is too large, but could be computationally costly if it is too small. The issue is that one population's update rates occur on vastly different scales than the other's, hence the timing of respective events occur on separate scales. This issue is analogous to the problem where our tumor population decreases rapidly, but both the normal and CAR T cell populations remain relatively high for some time. Therefore, we here propose a method that exploits this separation of time scales, to produce a fast algorithm, while maintaining the fluctuations necessary for complete response.

Hybrid Model

Hybrid models that combine the speed of deterministic models with the accuracy of stochastic models has been used before in several different contexts [15, 16, 17]. Here, we develop a hybrid model for our four population system. The process involves communicating information about the population between both models and using them when appropriate. We will need to keep track of the discrete and continuous population sizes (to convert from continuous to discrete, we use the ceiling function). All peripheral measurements of cells/μL were converted to total T cell counts assuming, 5 L of blood and multiplying by 10⁸, under the assumption that 1% of cells are in periphery at all times and converting μL to L. The final piece needed to connect the two is to define the threshold at which one considers fluctuations important. We set the stochastic threshold S to be 100 cells and we show in section “Parameter variability, virtual patient cohorts, and stochastic thresholds sensitivity” that this threshold does not impact patient outcomes for S=10−1000. An outline of our procedure is given below:

Define patient-level parameters (e.g. tumor growth rate, initial levels of CAR and normal T cells)

Initialize the discrete and continuous population vectors.

Set t=0.0 and define the final time to run the simulation (we typically used t=300−1000 measured in days).

While t<t_{final}:

Solve the ODE forward in time until the tumor population goes below the stochastic threshold (e.g. 100 cells) or the final time is reached.

Update the new continuous and discrete populations.

If a discrete (stochastic) event occurred, update the populations.

Determine the time till next stochastic event r and simulate the deterministic system until this next event (or t_{final}) is reached.

This method allows us to utilize the speed of simulating an ODE for the large populations and allows us to catch the complete response observed when the tumor population stochastically goes extinct. Parameter variability, virtual patient cohorts, and stochastic threshold sensitivity.

Parameter Variability, Virtual Patient Cohorts, and Stochastic Threshold Sensitivity

Using the parameter values reported in Table 1, we created additional parameter variability in the form of a mixture model/perturbation parameter a: We assumed that each parameter varies for each patient around the median with a normal distribution with mean equal to the median and variance equal to a times median.

To generate survival data in FIGS. 2A-4C, we ran the hybrid model for 1000-10000 computer-simulated instances, each with a potentially different set of parameters chosen from a distribution around the median parameter value. We then recorded the time and rate of cure or progression for each patient and parameter set. The Kaplan-Meier (K-M) curves were generated in Wolfram Mathematica, using the function SurvivalModelFit.

The perturbation parameter σ that governed the variability of all fitted parameters. In another sense, σ can be regarded as a sensitivity analysis parameter. Consider a parameter α, then the perturbed values can be in the range α[1+σU (−1, 1)], where U (a, b) is a uniform distribution with bounds a, b. From a clinical trial standpoint, we can consider a as a crude measure of patient variability, however without individual patient resolution. For example, if we wish to simulate a single patient to determine that individual's ‘optimal’ treatment as predicted from the median set of parameters fitted, we would choose σ=0. If we want to generate a cohort, we consider σ>0. For most analyses, we chose a range of σ=0.05−0.15.

The hybrid model adds an additional exogenous parameter to the system—the stochastic threshold S. Clearly, as S→0, the system approaches the full ODE model, while if S→∞ the system will be fully stochastic. In our simulations we chose S=100, but we investigated the sensitivity of the results for different S∈[10, 1000] and found that our results are robust to the threshold, making the hybrid model a valid approach for solving these problems without compromising speed by using a fully stochastic model. To test this, we generated a virtual cohort of 1000 patients and compared the rate of cure, mean time of cure and progression distributions against different values of S.

We also calculated the mean cure time and progression time as a function of S. From this we obtained the coefficient of variations for cure c_{cure}=σ/μ˜0.036 and c_{progression}˜0.018. This shows that the variability due to changing S changed these quantities by 1.8-3.6% of their respective means. The empirical distributions were also tested using the non-parametric K-S and Pearson tests. We considered S=100 our null distribution and compared it against all the other distributions (both cure and progression time). All comparisons and both tests yielded p-values above 5%, with a median p-value of around 50%. This is a strong indicator that these came from the same underlying distribution.

Thus we can reasonably conclude that the results are not sensitive to the choice of S.

Progressive Disease Criterion and its Impact on K-S Curves

For simulations, we defined PD to occur when the tumor size first exceeds a threshold ß related to the initial tumor burden B₀. That is t_{progression} was defined as the time when B>ßB₀. Clinical progression defines ß=1.5, while in the simulations, we took ß=2.0. The point of taking a higher threshold is to avoid early termination of the code, which exits upon a patient exceeding the threshold given or when B=0, the patient is cured. In theory, the threshold impacts when a person progresses and has no impact on the outcome. We can estimate this effect by conditioning on patients who enter the stochastic region and assume that once they leave it the impact of CAR on its growth is negligible. Then the timing to progression once a patient leaves stochastic threshold reduces to solving ßB₀=Se^{r}B tprogression. Solving this leads to

$${t}_{progression} ( β , S ) = \frac{1}{{r}_{B}} ln ( \frac{β {B}_{0}}{S} ) . \qquad ( 37 )$$

Comparing relative times ß_{clinical}, ßₛᵢₘᵤₗₐₜᵢₒₙ leads to

$${t}_{progression} ( {β}_{simulation} , S ) = \frac{ln ( \frac{{β}_{simulation} {B}_{0}}{S} )}{ln ( \frac{{β}_{clinical} {B}_{0}}{S} )} {t}_{progression} ( {β}_{clinical} , S ) . \qquad ( 38 )$$

With S=100, ß_{clinical}=1.5, B₀=2×10¹¹, we see the relationship

t_{progression}(βₛᵢₘᵤₗₐₜᵢₒₙ,100)=(0.981+0.046 ln βₛᵢₘᵤₗₐₜᵢₒₙ)t_{progression}(1.5,100) (39)

The impact of the threshold criterion on the progression time is logarithmic. For our threshold value ßₛᵢₘᵤₗₐₜᵢₒₙ=2, we see that our simulation delays calling progression by about 1% compared with the clinical cutoff. Using a threshold value ßₛᵢₘᵤₗₐₜᵢₒₙ=5 is about 5.5%. This idea can be generalized to any initial starting value (not just 5) at which B′≈r_{B}B (e.g. for tumors that don't make it to the stochastic region). In FIGS. 8A-8C, we show the predicted shift in when progression time occurs (gray curves). For the most part, it correctly encapsulates the change from switching thresholds, albeit for predicting earlier progression between days 50-80. This can be explained by noting that not all simulations necessarily enter the stochastic region, thus the initial value problem we solved to obtain our estimated change in progression time will be more sensitive to B. This is seen by noting that B₀/S»ß, but as our initial value becomes closer to B₀ (e.g. if a patient does not enter the stochastic region), then ß becomes more relevant. Again, we stress that we are only talking about the progression time, as we can see in the simulations the PFS approaches the same values for all cutoffs.

FIGS. 8A-8C also highlights the potential error with choosing a ß that is too small. For example in FIG. 8 C, ß=1.5 shows a dramatic early decline from days 0 to around 5. These patients progressed because they reached the progression threshold. Interestingly, not all these “progressors” actually progressed as can be seen by the following argument. Let [symbol] P_{ß=1.5}⁺(t)) be the probability that a person is deemed to have progressed by time t with ß=1.5 cutoff. Define [symbol] (T(t)) to be the true probability of progression by time t. We want to know the number of true progressions that occurred during the initial dip in the PFS curve. Mathematically,

$$ℙ ( T ( {t}_{f} ) \vert {P}_{β = 1.5}^{+} ( t < 5 ) ) = \frac{ℙ ( {P}_{β = 1.5}^{+} ( t < 5 ) , T ( {t}_{f} ) )}{ℙ ( {P}_{β = 1.5}^{+} ( t < 5 ) )} = \frac{ℙ ( T ( {t}_{f} ) ) - ℙ ( T ( {t}_{f} ) , {P}_{β = 1.5}^{+} ( t \geq 5 ) )}{ℙ ( {P}_{β = 1.5}^{+} ( t < 5 ) )} . \qquad ( 40 )$$

We can estimate from the PFS curve that [symbol] (T(t_{f}))≈0.594 [symbol] T(t_{f}), P_{ß=1.5}⁺(t>5))≈0.569 is inferred from conditioning out the patients who progressed early (it is related to the long-term PFS of the purple curve). Finally, from the unconditioned PFS curve, we have [symbol] (P_{ß=1.5}⁺)(t<5))=0.083. Plugging into our equation above yields

[symbol]

(T(t_{f})|P_{β=1.5}⁺(t<5))=0.30. (41)

Hence, only 30% of the simulated patients who progressed in the lower cutoff would truly have progressed, implying that 70% of those were actually going to eventually become cured! To avoid this potential pitfall, we used ß=2.0 in all numerical simulations in the main text, as FIGS. 8A-8C demonstrates that the shift in progression time is minimal and larger ß's will avoid this potential numerical pitfall.

### REFERENCES

### Example 1

[1] Turtle, C. J. et al. CD19 CAR-T cells of defined CD4+: CD8+ composition in adult B cell ALL patients. The Journal of Clinical Investigation 126, 2123-2138 (2016).
[2] Howlader, N. et al. SEER Cancer Statistics Review, 1975-2014. (2014).
[3] Crump, M. et al. Outcomes in refractory diffuse large B-cell lymphoma: results from the international SCHOLAR-1 study. Blood 130, 1800-1808, doi:10.1182/blood-2017-03-769620 (2017).
[4] Rich, R. R. et al. Clinical immunology: Principles and Practice. Fourth edn, (Saunders, London, U K, 2013).
[5] Locke, F. L. et al. Long-term safety and activity of axicabtagene ciloleucel in refractory large B-cell lymphoma (ZUMA-1): a single-arm, multicentre, phase 1-2 trial. Lancet Oncol 20, 31-42, doi:10.1016/51470-2045(18)30864-7 (2019).
[6] Locke, F. L. et al. Phase 1 Results of ZUMA-1: A Multicenter Study of KTE-C19 Anti-CD19 CAR T Cell Therapy in Refractory Aggressive Lymphoma. Mol Ther 25, 285-295, doi:10.1016/j.ymthe.2016.10.020 (2017).
[7] Neelapu, S. S. et al. Axicabtagene Ciloleucel CAR T-Cell Therapy in Refractory Large B-Cell Lymphoma. N Engl J Med 377, 2531-2544, doi:10.1056/NEJMoa1707447 (2017).
[8] Stein, A. M. et al. Tisagenlecleucel Model-Based Cellular Kinetic Analysis of Chimeric Antigen Receptor-T Cells. CPT Pharmacometrics Syst Pharmacol 8, 285-295, doi:10.1002/psp4.12388 (2019).
[⁹] Glodde, N. et al. Experimental and stochastic models of melanoma T-cell therapy define impact of subclone fitness on selection of antigen loss variants. biorxiv.org, https://doi.org/10.1101/860023 (2019).
[10] Sahoo, P. et al. Mathematical deconvolution of CAR T-cell proliferation and exhaustion from real-time killing assay data. J R Soc Interface 17, 20190734, doi:10.1098/rsif.2019.0734 (2020).
[11] Cess, C. G. & Finley, S. D. Data-driven analysis of a mechanistic model of CAR T cell signaling predicts effects of cell-to-cell heterogeneity. J Theor Biol 489, 110125, doi:10.1016/j.jtbi.2019.110125 (2019).
[12] Maude, S. L. et al. Tisagenlecleucel in Children and Young Adults with B-Cell Lymphoblastic Leukemia. N Engl J Med 378, 439-448, doi:10.1056/NEJMoa1709866 (2018).
[13] Henning, A. N., Klebanoff, C. A. & Restifo, N. P. Silencing stemness in T cell differentiation. Science 359, 163-164, doi:10.1126/science.aar5541 (2018).
[14] Pace, L. et al. The epigenetic control of stemness in CD8(+) T cell fate commitment. Science 359, 177-186, doi:10.1126/science.aah6499 (2018).
[15] Restifo, N. P. & Gattinoni, L. Lineage relationship of effector and memory T cells. Curr Opin Immunol 25, 556-563, doi:10.1016/j.coi.2013.09.003 (2013).
[16] Farber, D. L., Yudanin, N. A. & Restifo, N. P. Human memory T cells: generation, compartmentalization and homeostasis. Nat Rev Immunol 14, 24-35, doi:10.1038/nri3567 (2014).
[17] Locke, F. L. et al. Immune signatures of cytokine release syndrome and neurologic events in a multicenter registrational trial (ZUMA-1) in subjects with refractory diffuse large B cell lymphoma treated with axicabtagene ciloleucel (KTE-C19) [abstract]. Cancer Research 77, CT020, doi:doi:10.1158/1538-7445.AM2017-CT020 (2017).
[18] Turtle, C. J. et al. Immunotherapy of non-Hodgkin's lymphoma with a defined ratio of CD8+ and CD4+ CD19-specific chimeric antigen receptor-modified T cells. Sci Transl Med 8, 355ra116, doi:10.1126/scitranslmed.aaf8621 (2016).
[19] Brudno, J. N. et al. Safety and feasibility of anti-CD19 CAR T cells with fully human binding domains in patients with B-cell lymphoma. Nat Med 26, 270-280, doi:10.1038/s41591-019-0737-3 (2020).
[20] Gilpin, M. E. Minimal viable populations: processes of species extinction. Conservation biology: the science of scarcity and diversity (edited by M. E. Soulé) (1986).
[21] Sommermeyer, D. et al. Fully human CD19-specific chimeric antigen receptors for T-cell therapy. Leukemia 31, 2191-2199, doi:10.1038/Ieu.2017.57 (2017).
[22] Brady, R. & Enderling, H. Mathematical Models of Cancer: When to Predict Novel Therapies, and When Not to. Bull Math Biol 81, 3722-3731, doi:10.1007/s11538-019-00640-x (2019).
[23] Kochenderfer, J. N. et al. Lymphoma Remissions Caused by Anti-CD19 Chimeric Antigen Receptor T Cells Are Associated With High Serum Interleukin-15 Levels. J Clin Oncol 35, 1803-1813, doi:10.1200/JCO.2016.71.3024 (2017).
[24] Prokopiou, S. et al. A proliferation saturation index to predict radiation response and personalize radiotherapy fractionation. Radiation Oncology 10, 159 (2015).
[25] Poleszczuk, J. et al. Predicting patient-specific radiotherapy protocols based on mathematical model choice for Proliferation Saturation Index. Bulletin of Mathematical Biology 80, 1195-1206 (2017).
[26] Altrock, P. M., Liu, L. L. & Michor, F. The mathematics of cancer: integrating quantitative models. Nature Reviews Cancer 15, 730-745, doi:10.1038/nrc4029 (2015).
[27] Werner, B., Dingli, D., Lenaerts, T., Pacheco, J. M. & Traulsen, A. Dynamics of mutant cells in hierarchical organized tissues. PLoS Comput Biol 7, e1002290, doi:10.1371/journal.pcbi.1002290 (2011).
[28] Werner, B. et al. The Cancer Stem Cell Fraction in Hierarchically Organized Tumors Can Be Estimated Using Mathematical Modeling and Patient-Specific Treatment Trajectories. Cancer Res 76, 1705-1713, doi:10.1158/0008-5472.CAN-15-2069 (2016).
[29] Riddell, S. R. et al. T-cell mediated rejection of gene-modified HIV-specific cytotoxic T lymphocytes in HIV-infected patients. Nat Med 2, 216-223, doi:10.1038/nm0296-216 (1996).
[30] Jensen, M. C. et al. Antitransgene rejection responses contribute to attenuated persistence of adoptively transferred CD20/CD19-specific chimeric antigen receptor redirected T cells in humans. Biol Blood Marrow Transplant 16, 1245-1256, doi:10.1016/j.bbmt.2010.03.014 (2010).
[31] Neelapu, S. S. et al. Chimeric antigen receptor T-cell therapy—assessment and management of toxicities. Nat Rev Clin Oncol 15, 47-62, doi:10.1038/nrclinonc.2017.148 (2018).
[32] Lee, D. W. et al. ASTCT Consensus Grading for Cytokine Release Syndrome and Neurologic Toxicity Associated with Immune Effector Cells. Biol Blood Marrow Transplant 25, 625-638, doi:10.1016/j.bbmt.2018.12.758 (2019).
[33] Dholaria, B. R., Bachmeier, C. A. & Locke, F. Mechanisms and Management of Chimeric Antigen Receptor T-Cell Therapy-Related Toxicities. BioDrugs 33, 45-60, doi:10.1007/s40259-018-0324-z (2019).
[34] Jacobson, C. A. CD19 Chimeric Antigen Receptor Therapy for Refractory Aggressive B-Cell Lymphoma. J Clin Oncol 37, 328-335, doi:10.1200/JCO.18.01457 (2019).
[35] Turtle, C. J. et al. CD19 CAR-T cells of defined CD4+:CD8+ composition in adult B cell ALL patients. J Clin Invest 126, 2123-2138, doi:10.1172/jci85309 (2016).
[36] Park, J. H. et al. Long-Term Follow-up of CD19 CAR Therapy in Acute Lymphoblastic Leukemia. N Engl J Med 378, 449-459, doi:10.1056/NEJMoa1709919 (2018).
[37] Roesch, K., Hasenclever, D. & Scholz, M. Modelling lymphoma therapy and outcome. Bull Math Biol 76, 401-430, doi:10.1007/s11538-013-9925-3 (2014).

### Example 2

[1] W. F. Fagan and E. E. Holmes. Quantifying the extinction vortex. Ecology Letters, 9:51-60, 2006. D. T. Gillespie. Exact stochastic simulation of coupled chemical reactions. The Journal of Physical Chemistry, 81(25):2340-2361, 1977.
[2] Tibor Antal and P L Krapivsky. Exact solution of a two-type branching process: models of tumor progression. Journal of Statistical Mechanics: Theory and Experiment, 2011: P08018, 2011.
[3] S. Strogatz. Nonlinear Dynamics and Chaos: With Applications to Physics, Biology, Chemistry, and Engineering (Studies in Nonlinearity). Westview Pr, 2000.
[4] Sattva S Neelapu, Frederick L Locke, Nancy L Bartlett, Lazaros J Lekakis, David B Miklos, Caron A Jacobson, Ira Braunschweig, Olalekan O Oluwole, Tanya Siddiqi, Yi Lin, et al. Axicabtagene ciloleucel car t-cell therapy in refractory large b-cell lymphoma. New England Journal of Medicine, 377(26):2531-2544, 2017.
[5] Christopher Rackauckas and Qing Nie. Differentialequations. jl—a performant and feature-rich ecosystem for solving differential equations in Julia. Journal of Open Research Software, 5(1), 2017.
[6] Patrick Kofod Mogensen and Asbjørn Nilsen Riseth. Optim: A mathematical optimization pack-age for Julia. Journal of Open Source Software, 3(24), 2018.
[7] Cameron J Turtle, La\ddot{ }ila-A\ddot{ }icha Hanafi, Carolina Berger, Theodore A Gooley, Sindhu Cherian, Michael Hudecek, Daniel Sommermeyer, Katherine Melville, Barbara Pender, Tanya M Budiarto, et al. Cd19 car-t cells of defined cd4+: Cd8+ composition in adult b cell all patients. The Journal of clinical investigation, 126(6):2123-2138, 2016.
[8] Andreas Mayer, Yaojun Zhang, Alan S. Perelson, and Ned S. Wingreen.
Regulation oft cell expansion by antigen presentation dynamics. Proceedings of the National Academy of Sciences, 116(13):5914-5919, 2019.
[9] Zvi Grossman, Booki Min, Martin Meier-Schellersheim, and William E Paul. Concomitantregu-lation oft-cell activation and homeostasis. Nature Reviews Immunology, 4(5):387, 2004.
[10] Karolina Pilipow, Alessandra Roberto, Mario Roederer, Thomas A Waldmann, Domenico Mavilio, and Enrico Lugli. il15 and t-cell stemness in t-cell-based cancer immunotherapy. Cancer research, 75(24):5187-5193, 2015.
[11] Katja Roesch, Dirk Hasenclever, and Markus Scholz. Modelling lymphoma therapy and outcome.
   Bulletin of Mathematical Biology, 76(2):401-430, 2014.
[12] M. E. Gilpin. Minimum viable populations: Processes of species extinction. In Conservation Biology: The Science of Scarcity and Diversity (edited by M. E. Soulé), pages 19-34. Sinauer, Sunderland, Mass., 1986.
[13] Daniel T Gillespie. Approximate accelerated stochastic simulation of chemically reacting systems.
   The Journal of Chemical Physics, 115(4):1716-1733, 2001.
[14] Fabian Spill, Pilar Guerrero, Tomas Alarcon, Philip K Maini, and Helen Byrne. Hybrid ap-proaches for multiple-species stochastic reaction—diffusion models. Journal of computational physics, 299:429-445, 2015.
[15] Karla Misselbeck, Luca Marchetti, Martha S Field, Marco Scotti, Corrado Priami, and Patrick J Stover. A hybrid stochastic model of folate-mediated one-carbon metabolism: Effect of the com-mon c677t mthfr variant on de novo thymidylate biosynthesis. Scientific reports, 7(1):797, 2017. Aurélien Alfonsi, Eric Cances, Gabriel Turinici, Barbara Di Ventura, and Wilhelm Huisinga. Adaptive simulation of hybrid stochastic and deterministic models for biochemical systems. In ESAIM: proceedings, volume 14, pages 1-13. EDP Sciences, 2005.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer-implemented method, comprising:
generating a model configured to represent dynamics and interactions among normal T cells, engineered cells, and tumor cells, wherein the model comprises a plurality of cell population compartments;
receiving pre-treatment patient data for a cancer patient;
receiving post-treatment patient data for the cancer patient, wherein each of the pre-treatment patient data and the post-treatment patient data comprise a measure of at least one of tumor volume, total lymphocytes, memory T cells, memory engineered cells, tumor killing cells, or antigen-presenting tumor cells; and
quantitatively predicting the cancer patient's response to an immune-based or targeted therapy using the model, the pre-treatment patient data, and the post-treatment patient data.

2. The computer-implemented method of claim 1, wherein the model is configured to simulate interactions between normal T cells and engineered cells.

3. The computer-implemented method of claim 1, wherein the model is configured to simulate a differentiation rate of memory engineered cells to tumor killing cells.

4. The computer-implemented method claim 1, wherein the plurality of cell population compartments comprise normal naïve/memory T cells, naïve/memory engineered cells, tumor killing cells, and antigen-presenting tumor cells.

5. The computer-implemented method of claim 4, wherein the plurality of cell population compartments are modelled based on continuous-time birth and death stochastic processes and deterministic mean-field equations.

6. The computer-implemented method of claim 1, wherein the post-treatment patient data further comprises a measure of at least one tumor growth rate, tumor cell extinction rate, memory T cell recovery rate, naïve/memory engineered cell expansion rate, naïve/memory engineered cell differentiation rate, tumor killing cell death rate, or tumor killing cell exhaustion rate.

7. The computer-implemented method of claim 1, wherein the quantitative prediction of the cancer patient's response to the immune-based or targeted therapy is a probability of tumor extinction.

8. The computer-implemented method of claim 7, wherein the probability of tumor extinction is predicted for a fixed point in time.

9. The computer-implemented method of claim 7, wherein the probability of tumor extension is predicted over a range of time.

10. The computer-implemented method of claim 1, wherein the quantitative prediction of the cancer patient's response to the immune-based or targeted therapy is a progression-free survival (PFS).

11. The computer-implemented method of claim 1, wherein the pre-treatment patient data is derived from a blood or tissue sample obtained at a time of or before administration of the immune-based or targeted therapy to the cancer patient.

12. The computer-implemented method of claim 1, wherein the post-treatment patient data is derived from a blood or tissue sample obtained at a time after administration of the immune-based or targeted therapy to the cancer patient.

13. The computer-implemented method of claim 1, wherein the engineered cells are chimeric antigen receptor (CAR) T cells.

14. The computer-implemented method of claim 13, wherein each of the pre-treatment patient data and the post-treatment patient data comprise a measure of at least one of tumor volume, total lymphocytes, memory T cells, memory CAR T cells, effector CAR T cells, or antigen-presenting tumor cells.

15. A method, comprising:
receiving pre-treatment patient data for a cancer patient;
administering an immune-based or targeted therapy to the cancer patient;
receiving post-treatment patient data for the cancer patient, wherein each of the pre-treatment patient data and the post-treatment patient data comprise a measure of at least one of tumor volume, total lymphocytes, memory T cells, memory engineered cells, tumor killing cells, or antigen-presenting tumor cells;
quantitatively predicting the cancer patient's response to the immune-based or targeted therapy using a model, the pre-treatment patient data, and the post-treatment patient data, wherein the model is configured to represent dynamics and interactions among normal T cells, engineered cells, and tumor cells, and wherein the model comprises a plurality of cell population compartments;
adjusting the immune-based or targeted therapy based upon the quantitative prediction; and
administering the adjusted immune-based or targeted therapy to the cancer patient.

16. The method of claim 15, wherein the engineered cells are chimeric antigen receptor (CAR) T cells.

17. The method of claim 16, wherein each of the pre-treatment patient data and the post-treatment patient data comprise a measure of at least one of tumor volume, total lymphocytes, memory T cells, memory CAR T cells, effector CAR T cells, or antigen-presenting tumor cells.

18. A system, comprising:
a processor; and
a memory operably coupled to the processor, the memory having computer-executable instructions stored thereon that, when executed by the processor, cause the processor to:
generate a model configured to represent dynamics and interactions among normal T cells, engineered cells, and tumor cells, wherein the model comprises a plurality of cell population compartments;
receive pre-treatment patient data for a cancer patient;
receive post-treatment patient data for the cancer patient, wherein each of the pre-treatment patient data and the post-treatment patient data comprise a measure of at least one of tumor volume, total lymphocytes, memory T cells, memory engineered cells, tumor killing cells, or antigen-presenting tumor cells; and
quantitatively predict the cancer patient's response to an immune-based or targeted therapy using the model, the pre-treatment patient data, and the post-treatment patient data.

19. The system of claim 18, wherein the engineered cells are chimeric antigen receptor (CAR) T cells.

20. The system of claim 19, wherein each of the pre-treatment patient data and the post-treatment patient data comprise a measure of at least one of tumor volume, total lymphocytes, memory T cells, memory CAR T cells, effector CAR T cells, or antigen-presenting tumor cells.

21-30. (canceled)
